(19) United States
(12) Patent Application Publication
(10) Pub. No.: US 2025/0368973 A1
(43) Pub. Date: Dec. 4, 2025

(54) HOMING ENDONUCLEASE VARIANTS

(71) Applicant: Novo Nordisk A/S, Bagsværd (DK)

(72) Inventors: Jordan JARJOUR, Seattle, WA (US); Kyle HAVENS, Seattle, WA (US); Constantine CHRYSOSTOMOU, Fairfax, VA (US)

(73) Assignee: Novo Nordisk A/S, Bagsværd (DK)

(21) Appl. No.: 19/303,729

(22) Filed: Aug. 19, 2025

Related U.S. Application Data
(63) Continuation of application No. 17/311,464, filed on Jun. 7, 2021, now Pat. No. 12,404,500.
(60) Provisional application No. 62/777,476, filed on Dec. 10, 2018.

(51) Int. Cl. C12N 9/22

(52) U.S. Cl. CPC C12N 9/22

(57) ABSTRACT

The present disclosure provides homing endonuclease variants and megaTALs reprogrammed to bind and cleave a polynucleotide sequence in the genome. The homing endonuclease variants and megaTALs have been engineered to increase the thermostability and/or activity.

### CROSS REFERENCE TO RELATED APPLICATIONS

This application is a continuation application of U.S. patent application Ser. No. 17/311,464, filed on Jun. 7, 2021, which is a U.S. National Stage Application pursuant to 35 U.S.C. § 371 of International Patent Application PCT/US2019/065211, filed on Dec. 9, 2019, which published as WO 2020/123371 on Jun. 18, 2020, which claims priority to U.S. Provisional Patent Application No. 62/777,476, filed on Dec. 10, 2018, all of which are incorporated herein by reference in their entireties for all purposes.

### STATEMENT REGARDING SEQUENCE LISTING

The Sequence Listing associated with this application is provided in XML format, and is hereby incorporated by reference into the specification. The name of the XML file containing the Sequence Listing is “689767.0175 (BLUE-110.C1) Sequence Listing.” The XML file is 91,226 bytes, was created on Aug. 19, 2025, and is being submitted electronically, concurrent with the filing of the specification.

### BACKGROUND

### Technical Field

The present disclosure relates to genome editing compositions with improved stability and activity. More particularly, the disclosure relates to nuclease variants with improved stability and/or activity, compositions, and methods of making and using the same for genome editing.

### Description of the Related Art

Mutations in 3000 human genes have already been linked to disease phenotypes (omim.org), and more disease relevant genetic variations are being uncovered at a staggeringly rapid pace, many of which are associated with monogenetic diseases or cancer. Genome editing strategies based on programmable nucleases such as meganucleases, zinc finger nucleases, transcription activator-like effector nucleases and the clustered regularly interspaced short palindromic repeat (CRISPR)-associated nuclease Cas9 hold tremendous, but as yet unrealized, potential for the treatment of diseases, disorders, and conditions with a genetic component. Particular obstacles to implementing nuclease-based genome editing tools as therapeutic strategies include, but are not limited to low genome editing efficiencies, nuclease specificity, nuclease stability, and delivery challenges. The current state of the art for most genome editing strategies fails to meet some or all of these criteria.

### BRIEF SUMMARY

The present disclosure generally relates, in part, to compositions comprising homing endonuclease (HE) variants and megaTALs with improved stability and activity that cleave a target site in the human genome and methods of using the same. In particular embodiments, the HE variants and megaTALs are engineered to improve or enhance the thermostability of the enzyme and/or improve the catalytic activity of the enzyme.

In various embodiments, the present disclosure contemplates, in part, a polypeptide comprising an engineered homing endonuclease that has been engineered to improve stability and binding and cleavage of a target site.

In various embodiments, an I-OnuI homing endonuclease (HE) variant comprises one or more amino acid substitutions relative to a parent I-OnuI HE comprising the amino acid sequence set forth in SEQ ID NO: 1, wherein the one or more amino acid substitutions increase the thermostability of the I-OnuI HE variant compared to the parent I-OnuI HE.

In certain embodiments, the one or more amino acid substitutions is at an amino acid position selected from the group consisting of: I14, A19, V116, F168, D208, N246, and L263.

In certain embodiments, the amino acid substitutions are at the following amino acid positions: I14, A19, F168, D208, and N246.

In some embodiments, the one or more amino acid substitutions is at an amino acid position selected from the group consisting of: K108, K156, S176, E231, V261, E277, and G300. In some embodiments, the one or more amino acid substitutions is at an amino acid position selected from the group consisting of: N31, N33, K52, Y97, K124, K147, I153, K209, E264, and D268.

In particular embodiments, an I-OnuI HE variant comprises three or more amino acid substitutions is at an amino acid position selected from the group consisting of: I14, A19, K108, V116, K156, F168, S176, D208, E231, N246, V261, L263, E277, and G300.

In particular embodiments, an I-OnuI HE variant comprises three or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

In further embodiments, the I-OnuI HE variant comprises five or more amino acid substitutions is at an amino acid position selected from the group consisting of: I14, A19, K108, V116, K156, F168, S176, D208, E231, N246, V261, L263, E277, and G300.

In certain embodiments, an I-OnuI HE variant comprises five or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

In particular embodiments, an I-OnuI HE variant has a TM₅₀ at least 10° C. higher than the TM₅₀ of the parent I-OnuI HE.

In some embodiments, an I-OnuI HE variant has a TM₅₀ at least 15° C. higher than the TM₅₀ of the parent I-OnuI HE.

In certain embodiments, an I-OnuI HE variant has a TM₅₀ at least 20° C. higher than the TM₅₀ of the parent I-OnuI HE.

In certain embodiments, an I-OnuI HE variant has a TM₅₀ at least 25° C. higher than the TM₅₀ of the parent I-OnuI HE.

In particular embodiments, an I-OnuI HE variant targets a site in a gene selected from the group consisting of: HBA, HBB, HBG1, HBG2, BCL11A, PCSK9, TCRA, TCRB, B2M, HLA-A, HLA-B, HLA-C, HLA-E, HLA-F, HLA-G, CIITA, AHR, PD-1, CTLA4, TIGIT, TGFBR2, LAG-3, TIM-3, BTLA, IL4R, IL6R, CXCR1, CXCR2, IL10R, IL13Ra2, TRAILR1, RCASIR, and FAS.

In various embodiments, an I-OnuI homing endonuclease (HE) variant comprising one or more amino acid substitutions relative to a parent I-OnuI HE sequence, wherein the one or more amino acid substitutions increase the thermostability of the I-OnuI HE variant compared to the parent I-OnuI HE.

In further embodiments, a parent I-OnuI HE amino acid sequence set is forth in SEQ ID NO: 1.

In further embodiments, the one or more amino acid substitutions is at an amino acid position selected from the group consisting of: I14, A19, V116, F168, D208, N246, and L263.

In certain embodiments, the amino acid substitutions are at the following amino acid positions: I14, A19, F168, D208, and N246.

In certain embodiments, the amino acid substituted for 114 is selected from the group consisting of: S, N, M, K, F, D, T, and V.

In some embodiments, the amino acid substituted for 114 is selected from the group consisting of: T and V.

In particular embodiments, the amino acid substituted for A19 is selected from the group consisting of: C, D, I, L, S, T and V.

In additional embodiments, the amino acid substituted for A19 is selected from the group consisting of: T and V.

In certain embodiments, the amino acid substituted for V116 is selected from the group consisting of: F, D, A, L and I.

In particular embodiments, the amino acid substituted for V116 is selected from the group consisting of: L and I.

In certain embodiments, the amino acid substituted for F168 is selected from the group consisting of: H, Y, I, V, P, L and S.

In additional embodiments, the amino acid substituted for F168 is selected from the group consisting of: L and S.

In some embodiments, the amino acid substituted for D208 is selected from the group consisting of: N, V, Y, and E.

In particular embodiments, the amino acid substituted for D208 is E.

In particular embodiments, the amino acid substituted for N246 is selected from the group consisting of: H, I, D, R, S, T, V, Y, and K.

In particular embodiments, the amino acid substituted for N246 is K.

In additional embodiments, the amino acid substituted for L263 is selected from the group consisting of: H, F, P, T, V, and R.

In further embodiments, the amino acid substituted for L263 is R.

In additional embodiments, the one or more amino acid substitutions is at an amino acid position selected from the group consisting of: K108, K156, S176, E231, V261, E277, and G300. In certain embodiments, the amino acid substituted for K108 is selected from the group consisting of: E, N, Q, R, T, V, and M.

In certain embodiments, the amino acid substituted for K108 is M

In some embodiments, the amino acid substituted for K156 is selected from the group consisting of: N, Q, R, T, V, I, and E.

In particular embodiments, the amino acid substituted for K156 is selected from the group consisting of: I and E

In additional embodiments, the amino acid substituted for S176 is selected from the group consisting of: P, N and A.

In further embodiments, the amino acid substituted for S176 is A.

In particular embodiments, the amino acid substituted for E231 is selected from the group consisting of: D, K, V, and G.

In particular embodiments, the amino acid substituted for E231 is selected from the group consisting of: K and G.

In certain embodiments, the amino acid substituted for V261 is selected from the group consisting of: D, G, I, L, S, T, and A.

In some embodiments, the amino acid substituted for V261 is A.

In certain embodiments, the amino acid substituted for E277 is selected from the group consisting of: A, D, G, Q, V, and K.

In additional embodiments, the amino acid substituted for E277 is K.

In further embodiments, the amino acid substituted for G300 is selected from the group consisting of: S, V, D, C, and R.

In particular embodiments, the amino acid substituted for G300 is R.

In additional embodiments, the one or more amino acid substitutions is at an amino acid position selected from the group consisting of: N31, N33, K52, Y97, K124, K147, I153, K209, E264, and D268.

In some embodiments, the amino acid substituted for N31 is selected from the group consisting of: D, H, I, R, K, S, T and Y.

In additional embodiments, the amino acid substituted for N31 is K.

In particular embodiments, the amino acid substituted for N33 is selected from the group consisting of: D, G, H, I, K, S, T and Y.

In particular embodiments, the amino acid substituted for N33 is K.

In certain embodiments, the amino acid substituted for K52 is selected from the group consisting of: Q, R, T, Y, N, E, and M.

In further embodiments, the amino acid substituted for K52 is M.

In particular embodiments, the amino acid substituted for Y97 is selected from the group consisting of: F, N and H.

In other embodiments, the amino acid substituted for Y97 is F.

In particular embodiments, the amino acid substituted for K124 is selected from the group consisting of: E, N, R and T.

In some embodiments, the amino acid substituted for K124 is N.

In particular embodiments, the amino acid substituted for K147 is selected from the group consisting of: E, I, N, R and T.

In certain embodiments, the amino acid substituted for K147 is I.

In further embodiments, the amino acid substituted for 1153 is selected from the group consisting of: D, H, K, T, Y, S, V and N.

In other embodiments, the amino acid substituted for 1153 is N.

In certain embodiments, the amino acid substituted for K209 is selected from the group consisting of: E, M, N, Q and R.

In particular embodiments, the amino acid substituted for K209 is R.

In additional embodiments, the amino acid substituted for E264 is selected from the group consisting of: A, D, G, K, Q, R and V.

In certain embodiments, the amino acid substituted for E264 is K.

In further embodiments, the amino acid substituted for D268 is selected from the group consisting of: A, E, G, H, N, V and Y.

In particular embodiments, the amino acid substituted for D268 is N.

In particular embodiments, an I-OnuI HE variant comprises three or more amino acid substitutions.

In additional embodiments, the I-OnuI HE variant comprises five or more amino acid substitutions.

In particular embodiments, the I-OnuI HE variant comprises three or more amino acid substitutions is at an amino acid position selected from the group consisting of: I14, A19, K108, V116, K156, F168, S176, D208, E231, N246, V261, L263, E277, and G300.

In further embodiments, an I-OnuI HE variant comprises three or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

In some embodiments, an I-OnuI HE variant comprises five or more amino acid substitutions is at an amino acid position selected from the group consisting of: I14, A19, K108, V116, K156, F168, S176, D208, E231, N246, V261, L263, E277, and G300.

In certain embodiments, an I-OnuI HE variant comprises five or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

In additional embodiments, an I-OnuI HE variant has a TM₅₀ at least 10° C. higher than the TM₅₀ of the parent I-OnuI HE.

In particular embodiments, an I-OnuI HE variant has a TM₅₀ at least 15° C. higher than the TM₅₀ of the parent I-OnuI HE.

In particular embodiments, an I-OnuI HE variant has a TM₅₀ at least 20° C. higher than the TM₅₀ of the parent I-OnuI HE.

In further embodiments, an I-OnuI HE variant has a TM₅₀ at least 25° C. higher than the TM₅₀ of the parent I-OnuI HE.

In particular embodiments, an I-OnuI HE variant targets a site in a gene selected from the group consisting of: HBA, HBB, HBG1, HBG2, BCL11A, PCSK9, TCRA, TCRB, B2M, HLA-A, HLA-B, HLA-C, HLA-E, HLA-F, HLA-G, CIITA, AHR, PD-1, CTLA4, TIGIT, TGFBR2, LAG-3, TIM-3, BTLA, ILAR, IL6R, CXCR1, CXCR2, IL10R, IL13Ra2, TRAILR1, RCASIR, and FAS.

In various embodiments, an I-OnuI homing endonuclease (HE) variant that cleaves a target site in the human BCL11A gene, comprises one or more amino acid substitutions relative to a parent I-OnuI HE sequence, wherein the one or more amino acid substitutions increase the thermostability of the I-OnuI HE variant compared to the parent I-OnuI HE.

In various embodiments, an I-OnuI homing endonuclease (HE) variant that cleaves a target site in the human PCSK9 gene, comprises one or more amino acid substitutions relative to a parent I-OnuI HE sequence, wherein the one or more amino acid substitutions increase the thermostability of the I-OnuI HE variant compared to the parent I-OnuI HE.

In particular embodiments, an I-OnuI homing endonuclease (HE) variant that cleaves a target site in the human PDCD-1 gene, comprises one or more amino acid substitutions relative to a parent I-OnuI HE sequence, wherein the one or more amino acid substitutions increase the thermostability of the I-OnuI HE variant compared to the parent I-OnuI HE.

In some embodiments, an I-OnuI homing endonuclease (HE) variant that cleaves a target site in the human TCRα gene, comprises one or more amino acid substitutions relative to a parent I-OnuI HE sequence, wherein the one or more amino acid substitutions increase the thermostability of the I-OnuI HE variant compared to the parent I-OnuI HE.

In further embodiments, an I-OnuI homing endonuclease (HE) variant that cleaves a target site in the human CBLB gene, comprises one or more amino acid substitutions relative to a parent I-OnuI HE sequence, wherein the one or more amino acid substitutions increase the thermostability of the I-OnuI HE variant compared to the parent I-OnuI HE.

In particular embodiments, an I-OnuI homing endonuclease (HE) variant that cleaves a target site in the human CTLA-4 gene, comprises one or more amino acid substitutions relative to a parent I-OnuI HE sequence, wherein the one or more amino acid substitutions increase the thermostability of the I-OnuI HE variant compared to the parent I-OnuI HE.

In certain embodiments, an I-OnuI homing endonuclease (HE) variant that cleaves a target site in the human TGFβRII gene, comprises one or more amino acid substitutions relative to a parent I-OnuI HE sequence, wherein the one or more amino acid substitutions increase the thermostability of the I-OnuI HE variant compared to the parent I-OnuI HE.

In additional embodiments, I-OnuI homing endonuclease (HE) variant that cleaves a target site in the human TIM3 gene, comprises one or more amino acid substitutions relative to a parent I-OnuI HE sequence, wherein the one or more amino acid substitutions increase the thermostability of the I-OnuI HE variant compared to the parent I-OnuI HE.

In particular embodiments, an I-OnuI HE variant comprises three or more amino acid substitutions is at an amino acid position selected from the group consisting of: I14, A19, K108, V116, K156, F168, S176, D208, E231, N246, V261, L263, E277, and G300.

In particular embodiments, an I-OnuI HE variant comprises the amino acid substitutions at the following amino acid positions: I14, A19, F168, D208, and N246.

In certain embodiments, an I-OnuI HE variant comprises three or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

In particular embodiments, an I-OnuI HE variant comprises five or more amino acid substitutions is at an amino acid position selected from the group consisting of: I14, A19, K108, V116, K156, F168, S176, D208, E231, N246, V261, L263, E277, and G300.

In further embodiments, an I-OnuI HE variant comprises five or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

In particular embodiments, an I-OnuI HE variant has a TM₅₀ at least 10° C. higher than the TM₅₀ of the parent I-OnuI HE.

In some embodiments, an I-OnuI HE variant has a TM₅₀ at least 15° C. higher than the TM₅₀ of the parent I-OnuI HE.

In certain embodiments, an I-OnuI HE variant has a TM₅₀ at least 20° C. higher than the TM₅₀ of the parent I-OnuI HE.

In particular embodiments, an I-OnuI HE variant has a TM₅₀ at least 25° C. higher than the TM₅₀ of the parent I-OnuI HE.

### BRIEF DESCRIPTION OF SEVERAL VIEWS OF THE DRAWINGS

FIG. 1 shows the melt curves for I-OnuI LHE, and I-OnuI LHE variants reprogrammed to target CBLB and TCRα using a yeast surface display-based assay.

FIG. 2 shows a stacked bar graph of the percent change at each position from eight I-OnuI LHE variants randomly mutagenized and sorted for greater stability. The positions with a percent change greater than 2 standard deviations above the average are identified.

FIG. 3A shows the melt curves for a parent BCL11A I-OnuI LHE variant endonuclease, a BCL11A I-OnuI LHE variant endonuclease with individual stabilizing point mutations (F168S, I14T, N246I, V261A), and a BCL11A I-OnuI LHE variant that was randomly mutagenized (stable HE variant).

FIG. 3B shows the melt curves for a parent BCL11A I-OnuI LHE variant endonuclease, a post-sorted population of BCL11A I-OnuI LHE variants generated from a randomly mutagenized library, and a post-sorted population of BCL11A I-OnuI LHE variants generated from focused mutagenesis of amino acid positions that influence stability.

FIG. 3C shows the melt curves for a parent BCL11A I-OnuI LHE variant endonuclease and a BCL11A I-OnuI LHE A5 thermostability variant.

FIG. 4A-4C shows that the A5 thermostability enhancing mutations can stabilize I-OnuI LHE variants that target PCDC-1 (FIG. 4A), CBLB (FIG. 4B), and TCRα (FIG. 4C).

FIG. 5A shows a cartoon of a reporter construct used for assessing homing endonuclease stability.

FIG. 5B shows a western blot comparing protein expression of I-OnuI, a parental BCL11A I-OnuI LHE variant, and the BCL11A I-OnuI LHE A5 variant.

FIG. 5C shows a timecourse of expression of a GFP reporter compared to I-OnuI, a parental BCL11A I-OnuI LHE variant, and the BCL11A I-OnuI LHE A5 variant.

FIG. 6 shows that increasing thermostability of a PDCD-1 megaTAL significantly increases editing activity compared to its parent PDCD-1 megaTAL.

### BRIEF DESCRIPTION OF THE SEQUENCE IDENTIFIERS

SEQ ID NO: 1 is an amino acid sequence of a wild type I-OnuI LAGLIDADG homing endonuclease (LHE).

SEQ ID NO: 2 is an amino acid sequence of a wild type I-OnuI LHE.

SEQ ID NO: 3 is an amino acid sequence of a biologically active fragment of a wild-type I-OnuI LHE.

SEQ ID NO: 4 is an amino acid sequence of a biologically active fragment of a wild-type I-OnuI LHE.

SEQ ID NO: 5 is an amino acid sequence of a biologically active fragment of a wild-type I-OnuI LHE.

SEQ ID NO: 6 is an amino acid sequence of an I-OnuI LHE variant reprogrammed to bind and cleave a target site in the human TCRα gene.

SEQ ID NO: 7 is an amino acid sequence of an I-OnuI LHE variant reprogrammed to bind and cleave a target site in the human CBLB gene.

SEQ ID NO: 8 is an amino acid sequence of an I-OnuI LHE variant reprogrammed to bind and cleave a target site in the human BCL11A gene.

SEQ ID NOs: 9-14 set forth the amino acid sequences of I-OnuI LHE thermostable variants reprogrammed to bind and cleave a target site in the human BCL11A gene.

SEQ ID NO: 15 is an amino acid sequence of an I-OnuI LHE variant reprogrammed to bind and cleave a target site in the human PDCD-1 gene.

SEQ ID NO: 16 is an amino acid sequence of an I-OnuI LHE thermostable variant reprogrammed to bind and cleave a target site in the human PDCD-1 gene.

SEQ ID NO: 17 is an amino acid sequence of an I-OnuI LHE thermostable variant reprogrammed to bind and cleave a target site in the human TCRα gene.

SEQ ID NO: 18 is an amino acid sequence of an I-OnuI LHE thermostable variant reprogrammed to bind and cleave a target site in the human CBLB gene.

SEQ ID NO: 19 is an mRNA encoding a BCL11A I-OnuI HE variant.

SEQ ID NO: 20 is a codon optimized mRNA encoding a BCL11A I-OnuI HE variant.

SEQ ID NO: 21 is an mRNA encoding a BCL11A I-OnuI HE thermostable variant.

SEQ ID NO: 22 is an amino acid sequence that encodes a PDCD-1 megaTAL.

SEQ ID NO: 23 is an amino acid sequence that encodes a PDCD-1 megaTAL thermostable variant.

SEQ ID NOs: 24-34 set forth the amino acid sequences of various linkers.

SEQ ID NOs: 35-59 set forth the amino acid sequences of protease cleavage sites and self-cleaving polypeptide cleavage sites.

In the foregoing sequences, X, if present, refers to any amino acid or the absence of an amino acid.

### DETAILED DESCRIPTION

### A. Overview

The present disclosure generally relates to, in part, improved genome editing compositions and methods of use thereof. Genome editing enzymes hold tremendous promise for treating diseases, disorders, and conditions with a genetic component. To date, genome editing enzymes engineered to bind and cleave target sites in the genome may have short half-lives in vivo and/or fail to cleave with high efficiency. Without wishing to be bound to any particular theory, the inventors have discovered that homing endonuclease scaffolds can be engineered to increase thermostability and catalytic activity and that homing endonuclease activity unexpectedly increased when the enzymes were engineered to have greater thermostability. Moreover, the amino acid positions of homing endonucleases altered to increase thermostability and activity and reprogrammed to bind and cleave one target site are conserved and can be used to increase thermostability of other homing endonucleases reprogrammed to bind and cleave other target sites.

Genome editing compositions and methods contemplated in various embodiments comprise nuclease variants with enhanced stability and activity, designed to bind and cleave a target sequence present in a genome. The nuclease variants contemplated in particular embodiments, can be used to introduce a double-strand break in a target polynucleotide sequence, which may be repaired by non-homologous end joining (NHEJ) in the absence of a polynucleotide template, e.g., a donor repair template, or by homology directed repair (HDR), i.e., homologous recombination, in the presence of a donor repair template. Nuclease variants contemplated in certain embodiments, can also be designed as nickases, which generate single-stranded DNA breaks that can be repaired using the cell's base-excision-repair (BER) machinery or homologous recombination in the presence of a donor repair template. NHEJ is an error-prone process that frequently results in the formation of small insertions and deletions that disrupt gene function. Homologous recombination requires homologous DNA as a template for repair and can be leveraged to create a limitless variety of modifications specified by the introduction of donor DNA containing the desired sequence at the target site, flanked on either side by sequences bearing homology to regions flanking the target site.

In particular embodiments, homing endonucleases comprise one or more amino acid substitutions that increase stability and/or activity. In particular embodiments, homing endonucleases comprise 1, 2, 3, 4, 5, 6, 7, 8, 9, 10 or more amino acid substitutions that increase stability and/or activity.

In particular embodiments, homing endonucleases comprise one or more amino acid substitutions that increase stability and/or activity are formatted as a megaTAL. In particular embodiments, a megaTAL comprises a homing endonuclease that has 1, 2, 3, 4, 5, 6, 7, 8, 9, 10 or more amino acid substitutions that increase stability and/or activity.

In certain embodiments, compositions contemplated herein comprise a homing endonuclease variant or megaTAL that has been modified to increase stability and/or activity and optionally, an end-processing enzyme, e.g., Trex2.

In various embodiments, a cell or population of cells comprises a homing endonuclease variant or megaTAL that has been modified to increase stability and/or activity.

Accordingly, the methods and compositions contemplated herein represent a quantum improvement compared to existing adoptive cell therapies.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir andCC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives. As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured or modulated in a laboratory apparatus, usually under sterile conditions, and typically for a few hours or up to about 24 hours, but including up to 48 or 72 hours, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism. In one embodiment, cellular genomes are engineered, edited, or modified in vivo.

By “enhance” or “promote” or “increase” or “expand” or “potentiate” refers generally to the ability of a nuclease variant to produce, elicit, or cause a greater response (i.e., physiological response) compared to the response caused by either vehicle or control. A measurable response may include an increase in stability, e.g., thermostability, catalytic activity and/or binding affinity of a homing endonuclease variant relative to a parent homing endonuclease from which the variant was derived. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or control.

By “decrease” or “lower” or “lessen” or “reduce” or “abate” or “ablate” or “inhibit” or “dampen” refers generally to the ability of a nuclease variant contemplated herein to produce, elicit, or cause a lesser response (i.e., physiological response) compared to the response caused by either vehicle or control. A measurable response may include a decrease in lability, off-target binding affinity, off-target cleavage specificity, of a homing endonuclease variant relative to a parent homing endonuclease from which the variant was derived. A “decrease” or “reduced” amount is typically a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, or control.

By “maintain,” or “preserve,” or “maintenance,” or “no change,” or “no substantial change,” or “no substantial decrease” refers generally to the ability of a nuclease variant to produce, elicit, or cause a substantially similar or comparable physiological response (i.e., downstream effects) in as compared to the response caused by either vehicle or control. A comparable response is one that is not significantly different or measurable different from the reference response.

The terms “specific binding affinity” or “specifically binds” or “specifically bound” or “specific binding” or “specifically targets” as used herein, describe binding of one molecule to another, e.g., DNA binding domain of a polypeptide binding to DNA, at greater binding affinity than background binding. A binding domain “specifically binds” to a target site if it binds to or associates with a target site with an affinity or Kₐ (i.e., an equilibrium association constant of a particular binding interaction with units of 1/M) of, for example, greater than or equal to about 10⁵ M⁻¹. In certain embodiments, a binding domain binds to a target site with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (K_{d}) of a particular binding interaction with units of M (e.g., 10⁵ M to 10⁻¹³ M, or less). Affinities of nuclease variants comprising one or more DNA binding domains for DNA target sites contemplated in particular embodiments can be readily determined using conventional techniques, e.g., yeast cell surface display, or by binding association, or displacement assays using labeled ligands.

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

The terms “selectively binds” or “selectively bound” or “selectively binding” or “selectively targets” and describe preferential binding of one molecule to a target molecule (on-target binding) in the presence of a plurality of off-target molecules. In particular embodiments, an HE or megaTAL selectively binds an on-target DNA binding site about 5, 10, 15, 20, 25, 50, 100, or 1000 times more frequently than the HE or megaTAL binds an off-target DNA target binding site.

“On-target” refers to a target site sequence.

“Off-target” refers to a sequence similar to but not identical to a target site sequence.

A “target site” or “target sequence” is a chromosomal or extrachromosomal nucleic acid sequence that defines a portion of a nucleic acid to which a binding molecule will bind and/or cleave, provided sufficient conditions for binding and/or cleavage exist. When referring to a polynucleotide sequence or SEQ ID NO. that references only one strand of a target site or target sequence, it would be understood that the target site or target sequence bound and/or cleaved by a nuclease variant is double-stranded and comprises the reference sequence and its complement. In a preferred embodiment, the target site is a sequence in a human PDCD-1 gene.

“Protein stability” refers to the net balance of forces, which determine whether a protein will be its native folded conformation or a denatured (unfolded or extended) state. Protein unfolding, either partial or complete, can result in loss of function along with degradation by the cellular machinery. Polypeptide stability can be measured in response to various conditions including but not limited to temperature, pressure, and osmolyte concentration.

“Thermostability” refers to the ability of a protein to properly fold or remain in its native folded conformation and resist denaturation or unfolding upon exposure to temperature fluctuations. At non-ideal temperatures a protein will either not be able to efficiently fold into an active confirmation or will have the propensity to unfold from its active confirmation. A protein with increased thermostability will fold properly and retain activity over an increased range of temperatures when compared to a protein that is less thermostable.

“TM₅₀” refers to the temperature at which 50% of an amount of protein is unfolded. In particular embodiments, the TM₅₀ is the temperature at which an amount of protein has 50% maximum activity. In particular embodiments, TM₅₀ is a specific value determined by fitting multiple data points to a Boltzmann sigmoidal curve. In one non-limiting example, the TM₅₀ of a protein is measured in a yeast surface display activity assay by expressing the protein on the yeast surface at ˜25° C., aliquoting the yeast into multiple wells and exposing to a range of higher temperatures, cooling the yeast, and then measuring cleavage activity of the enzyme. As the temperature increases, more of the proteins lose their active confirmation, and therefore fewer protein expressing cells display sufficient activity to measure cleavage with flow cytometry. The temperature at which 50% of yeast display population is active as compared to the non-heat shocked population is the TM₅₀.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair (HDR) mechanisms. This process requires nucleotide sequence homology, uses a “donor” molecule as a template to repair a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part of or all of the sequence of the donor polynucleotide is incorporated into the target polynucleotide.

“NHEJ” or “non-homologous end joining” refers to the resolution of a double-strand break in the absence of a donor repair template or homologous sequence. NHEJ can result in insertions and deletions at the site of the break. NHEJ is mediated by several sub-pathways, each of which has distinct mutational consequences. The classical NHEJ pathway (cNHEJ) requires the KU/DNA-PKcs/Lig4/XRCC4 complex, ligates ends back together with minimal processing and often leads to precise repair of the break. Alternative NHEJ pathways (altNHEJ) also are active in resolving dsDNA breaks, but these pathways are considerably more mutagenic and often result in imprecise repair of the break marked by insertions and deletions. While not wishing to be bound to any particular theory, it is contemplated that modification of dsDNA breaks by end-processing enzymes, such as, for example, exonucleases, e.g., Trex2, may increase the likelihood of imprecise repair.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, polypeptides and nuclease variants, e.g., homing endonuclease variants, megaTALs, etc. contemplated herein are used for targeted double-stranded DNA cleavage. Endonuclease cleavage recognition sites may be on either DNA strand.

An “exogenous” molecule is a molecule that is not normally present in a cell, but that is introduced into a cell by one or more genetic, biochemical or other methods. Exemplary exogenous molecules include, but are not limited to small organic molecules, protein, nucleic acid, carbohydrate, lipid, glycoprotein, lipoprotein, polysaccharide, any modified derivative of the above molecules, or any complex comprising one or more of the above molecules. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, biopolymer nanoparticle, calcium phosphate co-precipitation, DEAE-dextran-mediated transfer and viral vector-mediated transfer.

An “endogenous” molecule is one that is normally present in a particular cell at a particular developmental stage under particular environmental conditions. Additional endogenous molecules can include proteins.

A “gene,” refers to a DNA region encoding a gene product, as well as all DNA regions which regulate the production of the gene product, whether or not such regulatory sequences are adjacent to coding and/or transcribed sequences. A gene includes, but is not limited to, promoter sequences, enhancers, silencers, insulators, boundary elements, terminators, polyadenylation sequences, post-transcription response elements, translational regulatory sequences such as ribosome binding sites and internal ribosome entry sites, replication origins, matrix attachment sites, and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

As used herein, the term “genetically engineered” or “genetically modified” refers to the chromosomal or extrachromosomal addition of extra genetic material in the form of DNA or RNA to the total genetic material in a cell. Genetic modifications may be targeted or non-targeted to a particular site in a cell's genome. In one embodiment, genetic modification is site specific. In one embodiment, genetic modification is not site specific.

As used herein, the term “genome editing” refers to the substitution, deletion, and/or introduction of genetic material at a target site in the cell's genome, which restores, corrects, disrupts, and/or modifies expression and/or function of a gene or gene product. Genome editing contemplated in particular embodiments comprises introducing one or more nuclease variants into a cell to generate DNA lesions at or proximal to a target site in the cell's genome, optionally in the presence of a donor repair template.

As used herein, the term “gene therapy” refers to the introduction of extra genetic material into the total genetic material in a cell that restores, corrects, or modifies expression of a gene or gene product, or for the purpose of expressing a therapeutic polypeptide. In particular embodiments, introduction of genetic material into the cell's genome by genome editing that restores, corrects, disrupts, or modifies expression of a gene or gene product, or for the purpose of expressing a therapeutic polypeptide is considered gene therapy.

### C. Nuclease Variants

Various engineered nucleases may lack sufficient stability to be used in a clinical setting. Nuclease variants contemplated herein have been modified to increase thermostability and enzymatic activity to enable clinical use of previously unstable enzymes. The nuclease variants are suitable for genome editing a target site and comprise one or more DNA binding domains and one or more DNA cleavage domains (e.g., one or more endonuclease and/or exonuclease domains), and optionally, one or more linkers contemplated herein. The engineered nucleases comprise one or amino acid substitutions that increase thermostability and/or activity compared to a reference or parent nuclease. The terms “reprogrammed nuclease,” “engineered nuclease,” or “nuclease variant” are used interchangeably and refer to a nuclease comprising one or more DNA binding domains and one or more DNA cleavage domains, wherein the nuclease has been designed to bind and cleave a double-stranded DNA target sequence and modified to increase thermostability and/or activity of the nuclease.

A “reference nuclease” or “parent nuclease” refers to a wild type nuclease, a nuclease found in nature, or a nuclease or variant that is modified to increase basal activity, affinity, specificity, selectivity, and/or stability to generate a subsequent nuclease variant.

In particular embodiments, a nuclease variant binds comprises at least 1 amino acid substitution that increases the stability and/or activity of the variant relative to a parent nuclease. In particular embodiments, a nuclease variant binds comprises at least 1, at least 2, at least 3, at least 4, at least 5, at least 6, at least 7, at least 8, at least 9, or at least 10 amino acid substitution that increases the stability and/or activity of the variant relative to a parent nuclease.

Nuclease variants may be designed and/or modified from a naturally occurring nuclease or from an existing nuclease variant. In preferred embodiments, a nuclease variant comprises increased thermostability and/or enzymatic activity compared to a parent nuclease variant. Nuclease variants contemplated in particular embodiments may further comprise one or more additional functional domains, e.g., an end-processing enzymatic domain of an end-processing enzyme that exhibits 5′-3′ exonuclease, 5′-3′ alkaline exonuclease, 3′-5′exonuclease (e.g., Trex2), 5′ flap endonuclease, helicase, template-dependent DNA polymerases or template-independent DNA polymerase activity.

Illustrative examples of nuclease variants reprogrammed to bind and cleave a target sequence and engineered to have increased thermostability include, but are not limited to, homing endonuclease (meganuclease) variants and megaTALs. In particular embodiments, the nuclease variants are reprogrammed to bind a target site or sequence in a gene selected from the group consisting of: HBA, HBB, HBG1, HBG2, BCL11A, PCSK9, TCRA, TCRB, B2M, HLA-A, HLA-B, HLA-C, HLA-E, HLA-F, HLA-G, CIITA, AHR, PD-1, CTLA4, TIGIT, TGFBR2, LAG-3, TIM-3, BTLA, IL4R, IL6R, CXCR1, CXCR2, IL10R, IL13Ra2, TRAILR1, RCASIR, and FAS.

### 1. Homing Endonuclease (Meganuclease) Variants

Homing endonucleases (meganucleases) are genome editing enzymes that can reprogrammed to bind and cleave selected target sites. However, some reprogrammed homing endonucleases were not sufficiently stable to allow further development or clinical use. The present inventors have unexpectedly discovered that certain amino acid positions in homing endonucleases affect stability, e.g., thermostability, of the enzymes; and further, substitution of amino acids at these positions can enhance enzyme stability compared to the parent enzyme, without sacrificing affinity or activity of the enzyme.

In various embodiments, a homing endonuclease or meganuclease is reprogrammed to introduce a double-strand break (DSB) in a target site and engineered to increase its thermostability, affinity, specificity, selectivity, and/or enzymatic activity. In preferred embodiments, a homing endonuclease is reprogrammed to bind and cleave a target site and engineered to increase the enzyme's thermostability relative to the thermostability of the enzyme from which it was designed.

“Homing endonuclease” and “meganuclease” are used interchangeably and refer to naturally-occurring homing endonucleases that recognize 12-45 base-pair cleavage sites and are commonly grouped into five families based on sequence and structure motifs: LAGLIDADG, GIY-YIG, HNH, His-Cys box, and PD-(D/E) XK.

A “reference homing endonuclease,” “reference meganuclease,” “parent homing endonuclease,” or “parent meganuclease” refers to a wild type homing endonuclease, a homing endonuclease found in nature, or a homing endonuclease that has been modified to increase basal activity, affinity, and/or stability to generate a subsequent homing endonuclease variant.

An “engineered homing endonuclease,” “reprogrammed homing endonuclease,” “homing endonuclease variant,” “engineered meganuclease,” “reprogrammed meganuclease,” or “meganuclease variant” refers to a homing endonuclease comprising one or more DNA binding domains and one or more DNA cleavage domains, wherein the homing endonuclease has been designed and/or modified from a parental or naturally occurring homing endonuclease to bind and cleave a DNA target sequence; has optionally undergone one or more rounds of refining affinity, selectivity, specificity, and/or activity; and has further been modified to have increased thermostability. Homing endonuclease variants may be designed and/or modified from a naturally occurring homing endonuclease or from another homing endonuclease variant. Homing endonuclease variants contemplated in particular embodiments may further comprise one or more additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase, template dependent DNA polymerase or template-independent DNA polymerase activity.

Homing endonuclease variants do not exist in nature and can be obtained by recombinant DNA technology or by random mutagenesis. Homing endonuclease variants may be obtained by making one or more amino acid alterations, e.g., mutating, substituting, adding, or deleting one or more amino acids, in a naturally occurring homing endonuclease or homing endonuclease variant. In particular embodiments, a homing endonuclease variant comprises one or more amino acid alterations to the DNA recognition interface to bind and cleave a selected target sequence and one or more amino acid substitutions to increase thermostability.

Homing endonuclease variants contemplated in particular embodiments may further comprise one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerase activity. In particular embodiments, homing endonuclease variants are introduced into a cell with an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerase activity. The homing endonuclease variant and 3′ processing enzyme may be introduced separately, e.g., in different vectors or separate mRNAs, or together, e.g., as a fusion protein, or in a polycistronic construct separated by a viral self-cleaving peptide or an IRES element.

A “DNA recognition interface” refers to the homing endonuclease amino acid residues that interact with nucleic acid target bases as well as those residues that are adjacent. For each homing endonuclease, the DNA recognition interface comprises an extensive network of side chain-to-side chain and side chain-to-DNA contacts, most of which is necessarily unique to recognize a particular nucleic acid target sequence. Thus, the amino acid sequence of the DNA recognition interface corresponding to a particular nucleic acid sequence varies significantly and is a feature of any natural or homing endonuclease variant. By way of non-limiting example, a homing endonuclease variant contemplated in particular embodiments may be derived by constructing libraries of HE variants in which one or more amino acid residues localized in the DNA recognition interface of the natural homing endonuclease (or a previously generated homing endonuclease variant) are varied. The libraries may be screened for target cleavage activity against each target site using cleavage assays (see e.g., Jarjour et al., 2009. Nuc. Acids Res. 37 (20): 6871-6880).

LAGLIDADG homing endonucleases (LHE) are the most well studied family of homing endonucleases, are primarily encoded in archaea and in organellar DNA in green algae and fungi, and display the highest overall DNA recognition specificity.

In one embodiment, a reprogrammed LHE or LHE variant that has been engineered to enhance thermostability is an I-OnuI HE variant (I-OnuI LHE variant). See e.g., SEQ ID NOs: 8-14 and 16-18.

In one embodiment, a reprogrammed I-OnuI HE or I-OnuI HE variant engineered to increase thermostability is generated from a natural I-OnuI, I-OnuI HE variant, or biologically active fragment thereof (e.g., SEQ ID NOs: 1-8 and 15). In preferred embodiments, a reprogrammed I-OnuI HE or I-OnuI HE variant engineered to increase thermostability is generated from an existing I-OnuI HE variant. In even more preferred embodiments, a reprogrammed I-OnuI HE or I-OnuI HE variant engineered to increase thermostability comprises 1, 2, 3, 4, 5, 6, 7, 8, 9, or 10 or more amino acid substitutions to increase thermostability of the enzyme compared to the thermostability of an existing parent I-OnuI HE variant.

In certain embodiments, an I-OnuI HE variant comprises at least 1, at least 2, at least 3, at least 4, at least 5, at least 6, or 7 amino acid substitutions of the following amino acid positions that have been identified to individually and collectively increase homing endonuclease thermostability: I14, A19, V116, F168, D208, N246, and L263 of representative I-OnuI amino acid sequences (SEQ ID NOs: 1-8 and 15), biologically active fragments thereof, and/or further variants thereof.

I In certain embodiments, an I-OnuI HE variant comprises amino acid substitutions of the following amino acid positions that have been identified to individually and collectively increase homing endonuclease thermostability: I14, A19, F168, D208, and N246 of representative I-OnuI amino acid sequences (SEQ ID NOs: 1-8 and 15), biologically active fragments thereof, and/or further variants thereof.

n particular embodiments, the amino acid substitution for 114 is selected from the group consisting of: 114S, I14N, 114M, 114K, I14F, I14D, I14T, and I14V. In preferred embodiments, the amino acid substitution for I14 is I14T or I14V. In particular embodiments, the amino acid substitution for A19 is selected from the group consisting of: A19C, A19D, A19I, A19L, A19S, A19T, and A19V. In preferred embodiments, the amino acid substitution for A19 is A19T or A19V. In particular embodiments, the amino acid substitution for V116 is selected from the group consisting of: V116F, V116D, V116A, V116L, and V116I. In preferred embodiments, the amino acid substitution for V116 is V116L or V116I. In particular embodiments, the amino acid substitution for F168 is selected from the group consisting of: F168H, F168Y, F168I, F168V, F168P, F168L, and F168S. In preferred embodiments, the amino acid substitution for F168 is F168L and F168S. In particular embodiments, the amino acid substitution for D208 is selected from the group consisting of: D208N, D208Y, D208V, and D208E. In preferred embodiments, the amino acid substitution for D208 is D208E. In preferred embodiments, the amino acid substitution for F168 is F168L, and F168S. In particular embodiments, the amino acid substitution for N246 is selected from the group consisting of: N246H, N246I, N246D, N246R, N246S, N246T, N246V, N246Y, and N246K. In preferred embodiments, the amino acid substitution for N246 is N246K. In particular embodiments, the amino acid substitution for L263 is selected from the group consisting of: L263H, L263F, L263P, L263T, L263V, and L263R. In preferred embodiments, the amino acid substitution for L263 is L263R.

In certain embodiments, an I-OnuI HE variant comprises at least 1, at least 2, at least 3, at least 4, at least 5, at least 6, or 7 amino acid substitutions of the following amino acid positions that have been identified to individually and collectively increase homing endonuclease thermostability: K108, K156, S176, E231, V261, E277, and G300 of representative I-OnuI amino acid sequences (SEQ ID NOs: 1-8 and 15), biologically active fragments thereof, and/or further variants thereof.

In particular embodiments, the amino acid substitution for K108 is selected from the group consisting of: K108E, K108N, K108Q, K108R, K108T, K108V, and K108M. In preferred embodiments, the amino acid substitution for K108 is K108M. In particular embodiments, the amino acid substitution for K156 is selected from the group consisting of: K156N, K156Q, K156R, K156T, K156V, K156I and K156E. In preferred embodiments, the amino acid substitution for K156 is K156I or K156E. In particular embodiments, the amino acid substitution for S176 is S176P, S176N or S176A. In preferred embodiments, the amino acid substitution for S176 is S176A. In particular embodiments, the amino acid substitution for E231 is selected from the group consisting of: E231D, E231V, E231K, and E231G. In preferred embodiments, the amino acid substitution for E231 is E231K or E231G. In particular embodiments, the amino acid substitution for V261 is selected from the group consisting of: V261D, V261G, V261I, V261L, V261S, V261T and V261A. In preferred embodiments, the amino acid substitution for V261 is V261A. In particular embodiments, the amino acid substitution for E277 is selected from the group consisting of: E277A, E277D, E277G, E277Q, E277V, and E277K. In preferred embodiments, the amino acid substitution for E277 is E277K. In particular embodiments, the amino acid substitution for G300 is selected from the group consisting of: G300S, G300V, G300D, G300C, and G300R. In preferred embodiments, the amino acid substitution for G300 is G300R.

Without wishing to be bound by any particular theory, the inventors have also discovered that each homing endonuclease reprogrammed to bind and cleave a particular target sequence may one or more additional amino acid positions that affect thermostability. In certain embodiments, an I-OnuI HE variant comprises at least 1, at least 2, at least 3, at least 4, at least 5, at least 6, at least 7, at least 8, at least 9, or 10 amino acid substitutions of the following amino acid positions that have been identified to individually, and in some cases collectively, increase homing endonuclease thermostability: N31, N33, K52, Y97, K124, K147, I153, K209, E264, and D268 of representative I-OnuI amino acid sequences (SEQ ID NOs: 1-8 and 15), biologically active fragments thereof, and/or further variants thereof.

In particular embodiments, the amino acid substitution for N31 is selected from the group consisting of: N31D, N31H, N31I, N31R, N31K, N31S, N31T and N31Y. In particular embodiments, the amino acid substitution for N31 is N31K. In particular embodiments, the amino acid substitution for N33 is selected from the group consisting of: N33D, N33G, N33H, N33I, N33K, N33S, N33T and N33Y. In particular embodiments, the amino acid substitution for N33 is N33K. In particular embodiments, the amino acid substitution for K52 is selected from the group consisting of: K52Q, K52R, K52T, K52Y, K52N, K52E, and K52M. In preferred embodiments, the amino acid substitution for K52 is K52M. In particular embodiments, the amino acid substitution for Y97 is selected from the group consisting of: Y97H, Y97N, and Y97F. In particular embodiments, the amino acid substitution for Y97 is Y97F. In particular embodiments, the amino acid substitution for K124 is selected from the group consisting of: K124E, K124N, K124R and K124T. In particular embodiments, the amino acid substitution for K124 is K124N. In particular embodiments, the amino acid substitution for K147 is selected from the group consisting of: K147E, K147I, K147N, K147R and K147T. In particular embodiments, the amino acid substitution for K147 is K147I. In particular embodiments, the amino acid substitution for I153 is selected from the group consisting of: I153D, I153H, I153K, I153T, I153Y, I153S, I153V and I153N. In preferred embodiments, the amino acid substitution for I153 is I153N. In particular embodiments, the amino acid substitution for K209 is selected from the group consisting of: K209E, K209M, K209N, K209Q and K209R. In particular embodiments, the amino acid substitution for K209 is K209R. In particular embodiments, the amino acid substitution for E264 is selected from the group consisting of: E264A, E264D, E264G, E264K, E264Q, E264R and E264V. In particular embodiments, the amino acid substitution for E264 is E264K. In particular embodiments, the amino acid substitution for D268 is selected from the group consisting of: D268A, D268E, D268G, D268H, D268N, D268V and D268Y. In particular embodiments, the amino acid substitution for D268 is D268N.

In particular embodiments, an I-OnuI HE variant comprises one or more, two or more, three or more, four or more, five or more, six or more, seven or more, eight or more, nine or more, 10 or more, 11 or more, 12 or more, 13 or more, or 14 amino acid substitutions of the following amino acid positions that have been identified to individually and collectively increase homing endonuclease thermostability: I14, A19, K108, V116, K156, F168, S176, D208, E231, N246, V261, L263, E277, and G300 of representative I-OnuI amino acid sequences (SEQ ID NOs: 1-8 and 15), biologically active fragments thereof, and/or further variants thereof.

In particular embodiments, an I-OnuI HE variant comprises one or more, two or more, three or more, four or more, five or more, six or more, seven or more, eight or more, nine or more, 10 or more, 11 or more, 12 or more, 13 or more, or 14 amino acid substitutions of the following amino acid positions that have been identified to individually and collectively increase homing endonuclease thermostability: I14, A19, K108, V116, K156, F168, S176, D208, E231, N246, V261, L263, E277, and G300; and one or more, two or more, three or more, four or more, five or more, six or more, seven or more, eight or more, nine or more, or 10 amino acid substitutions of the following amino acid positions that have been identified to individually, and in some cases collectively, increase homing endonuclease thermostability: N31, N33, K52, Y97, K124, K147, I153, K209, E264, and D268; of representative I-OnuI amino acid sequences (SEQ ID NOs: 1-8 and 15), biologically active fragments thereof, and/or further variants thereof.

In particular embodiments, an I-OnuI HE variant that binds and cleaves a target sequence comprises one or more, two or more, three or more, four or more, five or more, six or more, seven or more, eight or more, nine or more, or 10 amino acid substitutions to increase thermostability and is at least 85%, at least 86%, at least 87%, at least 88%, at least 89%, at least 90%, at least 91%, at least 92%, at least 93%, at least 94%, at least 95%, at least 96%, at least 97%, or at least 98% or at least 99% identical to the amino acid sequence set forth in any one of SEQ ID NOs: 1-18 or a biologically active fragment thereof.

In particular embodiments, an I-OnuI HE variant has increased thermostability compared to a parent I-OnuI LHE variant. In particular embodiments, an I-OnuI HE variant has a TM₅₀ about 5° C. higher to about 35° C. higher, about 10° C. higher to about 35° C. higher, about 10° C. higher to about 30° C. higher, about 10° C. higher to about 25° C. higher about 15° C. higher to about 35° C. higher about 15° C. higher to about 30° C., or about 15° C. higher to about 25° C. higher than the TM₅₀ of a parent I-OnuI HE or reference I-OnuI HE.

In particular embodiments, an I-OnuI HE variant has increased thermostability compared to a parent or reference I-OnuI LHE variant. In particular embodiments, an I-OnuI HE variant has a TM₅₀ about 5° C. higher, about 6° C. higher, about 7° C. higher, about 8° C. higher, about 9° C. higher, about 10° C. higher, about 11° C. higher, about 12° C. higher, about 13° C. higher, about 14° C. higher, about 15° C., about 16° C. higher, about 17° C. higher, about 18° C. higher, about 19° C. higher, about 20° C. higher, about 21° C. higher, about 22° C. higher, about 23° C. higher, about 24° C. higher, about 25° C. higher, about 26° C. higher, about 27° C. higher, about 28° C. higher, about 29° C. higher, about 30° C. higher, about 31° C. higher, about 32° C. higher, about 33° C. higher, about 34° C. higher, or about 35° C. higher than the TM₅₀ of a parent I-OnuI HE or reference I-OnuI HE.

In particular embodiments, an I-OnuI HE variant comprising one or more mutations to enhance thermostability is reprogrammed to bind a target site or sequence in a gene selected from the group consisting of: HBA, HBB, HBG1, HBG2, BCL11A, PCSK9, TCRA, TCRB, B2M, HLA-A, HLA-B, HLA-C, HLA-E, HLA-F, HLA-G, CIITA, AHR, PD-1, CTLA4, TIGIT, TGFBR2, LAG-3, TIM-3, BTLA, IL4R, IL6R, CXCR1, CXCR2, IL10R, IL13Ra2, TRAILR1, RCASIR, and FAS.

### 2. MegaTALs

MegaTALs are genome editing enzymes that combine the DNA binding properties of TAL DNA binding domains with the DNA binding and cleavage activities of homing endonucleases. Without wishing to be bound by any particular theory, it is believed that when a relatively unstable homing endonuclease is formatted as a megaTAL, the megaTAL is not inherently stabilized. Accordingly, introducing one or more stabilizing mutations into a homing endonuclease similarly stabilizes the corresponding megaTAL.

In various embodiments, a megaTAL comprises one or more TAL DNA binding domains and a homing endonuclease or meganuclease reprogrammed to introduce a double-strand break (DSB) in a target site and engineered to increase its thermostability, affinity, specificity, selectivity, and/or enzymatic activity of the enzyme. In preferred embodiments, the increased thermostability of a megaTAL comprising a homing endonuclease engineered to increase the enzyme's thermostability is relative to the thermostability of a megaTAL comprising the homing endonuclease before engineering to increase its thermostability.

A “megaTAL” refers to a polypeptide comprising a TALE DNA binding domain and a homing endonuclease variant that binds and cleaves a DNA target sequence and that has been engineered for increased thermostability, and optionally comprises one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase or template-independent DNA polymerase activity.

A “reference megaTAL” or “parent megaTAL” refers to a megaTAL that comprises a TALE DNA binding domain and a wild type homing endonuclease, a homing endonuclease found in nature, or a homing endonuclease that is modified to increase basal activity, affinity, and/or stability to generate a subsequent homing endonuclease variant.

In particular embodiments, a megaTAL can be introduced into a cell along with an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase, or template-independent DNA polymerase activity. The megaTAL and 3′ processing enzyme may be introduced separately, e.g., in different vectors or separate mRNAs, or together, e.g., as a fusion protein, or in a polycistronic construct separated by a viral self-cleaving peptide or an IRES element. A “TALE DNA binding domain” is the DNA binding portion of transcription activator-like effectors (TALE or TAL-effectors), which mimics plant transcriptional activators to manipulate the plant transcriptome (see e.g., Kay et al., 2007. Science 318:648-651). TALE DNA binding domains contemplated in particular embodiments are engineered de novo or from naturally occurring TALEs, e.g., AvrBs3 from Xanthomonas campestris pv. vesicatoria, Xanthomonas gardneri, Xanthomonas translucens, Xanthomonas axonopodis, Xanthomonas perforans, Xanthomonas alfalfa, Xanthomonas citri, Xanthomonas euvesicatoria, and Xanthomonas oryzae and brg11 and hpx17 from Ralstonia solanacearum. Illustrative examples of TALE proteins for deriving and designing DNA binding domains are disclosed in U.S. Pat. No. 9,017,967, and references cited therein, all of which are incorporated herein by reference in their entireties.

In particular embodiments, a megaTAL comprises a TALE DNA binding domain comprising one or more repeat units that are involved in binding of the TALE DNA binding domain to its corresponding target DNA sequence. A single “repeat unit” (also referred to as a “repeat”) is typically 33-35 amino acids in length. Each TALE DNA binding domain repeat unit includes 1 or 2 DNA-binding residues making up the Repeat Variable Di-Residue (RVD), typically at positions 12 and/or 13 of the repeat. The natural (canonical) code for DNA recognition of these TALE DNA binding domains has been determined such that an HD sequence at positions 12 and 13 leads to a binding to cytosine (C), NG binds to T, NI to A, NN binds to G or A, and NG binds to T. In certain embodiments, non-canonical (atypical) RVDs are contemplated.

Illustrative examples of non-canonical RVDs suitable for use in particular megaTALs contemplated in particular embodiments include, but are not limited to HH, KH, NH, NK, NQ, RH, RN, SS, NN, SN, KN for recognition of guanine (G); NI, KI, RI, HI, SI for recognition of adenine (A); NG, HG, KG, RG for recognition of thymine (T); RD, SD, HD, ND, KD, YG for recognition of cytosine (C); NV, HN for recognition of A or G; and H*, HA, KA, N*, NA, NC, NS, RA, S*for recognition of A or T or G or C, wherein (*) means that the amino acid at position 13 is absent. Additional illustrative examples of RVDs suitable for use in particular megaTALs contemplated in particular embodiments further include those disclosed in U.S. Pat. No. 8,614,092, which is incorporated herein by reference in its entirety.

In particular embodiments, a megaTAL contemplated herein comprises a TALE DNA binding domain comprising 3 to 30 repeat units. In certain embodiments, a megaTAL comprises 3, 4, 5, 6, 7, 8, 9, 10, 11, 12, 13, 14, 15, 16, 17, 18, 19, 20, 21, 22, 23, 24, 25, 26, 27, 28, 29, or 30 TALE DNA binding domain repeat units. In a preferred embodiment, a megaTAL contemplated herein comprises a TALE DNA binding domain comprising 5-15 repeat units, more preferably 7-15 repeat units, more preferably 9-15 repeat units, and more preferably 9, 10, 11, 12, 13, 14, or 15 repeat units.

In particular embodiments, a megaTAL contemplated herein comprises a TALE DNA binding domain comprising 3 to 30 repeat units and an additional single truncated TALE repeat unit comprising 20 amino acids located at the C-terminus of a set of TALE repeat units, i.e., an additional C-terminal half-TALE DNA binding domain repeat unit (amino acids −20 to −1 of the C-cap disclosed elsewhere herein, infra). Thus, in particular embodiments, a megaTAL contemplated herein comprises a TALE DNA binding domain comprising 3.5 to 30.5 repeat units. In certain embodiments, a megaTAL comprises 3.5, 4.5, 5.5, 6.5, 7.5, 8.5, 9.5, 10.5, 11.5, 12.5, 13.5, 14.5, 15.5, 16.5, 17.5, 18.5, 19.5, 20.5, 21.5, 22.5, 23.5, 24.5, 25.5, 26.5, 27.5, 28.5, 29.5, or 30.5 TALE DNA binding domain repeat units. In a preferred embodiment, a megaTAL contemplated herein comprises a TALE DNA binding domain comprising 5.5-15.5 repeat units, more preferably 7.5-15.5 repeat units, more preferably 9.5-15.5 repeat units, and more preferably 9.5, 10.5, 11.5, 12.5, 13.5, 14.5, or 15.5 repeat units.

In particular embodiments, a megaTAL comprises a TAL effector architecture comprising an “N-terminal domain (NTD)” polypeptide, one or more TALE repeat domains/units, a “C-terminal domain (CTD)” polypeptide, and a homing endonuclease variant. In some embodiments, the NTD, TALE repeats, and/or CTD domains are from the same species. In other embodiments, one or more of the NTD, TALE repeats, and/or CTD domains are from different species.

As used herein, the term “N-terminal domain (NTD)” polypeptide refers to the sequence that flanks the N-terminal portion or fragment of a naturally occurring TALE DNA binding domain. The NTD sequence, if present, may be of any length as long as the TALE DNA binding domain repeat units retain the ability to bind DNA. In particular embodiments, the NTD polypeptide comprises at least 120 to at least 140 or more amino acids N-terminal to the TALE DNA binding domain (0 is amino acid 1 of the most N-terminal repeat unit). In particular embodiments, the NTD polypeptide comprises at least about 120, 121, 122, 123, 124, 125, 126, 127, 128, 129, 130, 131, 132, 133, 134, 135, 136, 137, 138, 139, or at least 140 amino acids N-terminal to the TALE DNA binding domain. In one embodiment, a megaTAL contemplated herein comprises an NTD polypeptide of at least about amino acids +1 to +122 to at least about +1 to +137 of a Xanthomonas TALE protein (0 is amino acid 1 of the most N-terminal repeat unit). In particular embodiments, the NTD polypeptide comprises at least about 122, 123, 124, 125, 126, 127, 128, 129, 130, 131, 132, 133, 134, 135, 136, or 137 amino acids N-terminal to the TALE DNA binding domain of a Xanthomonas TALE protein. In one embodiment, a megaTAL contemplated herein comprises an NTD polypeptide of at least amino acids +1 to +121 of a Ralstonia TALE protein (0 is amino acid 1 of the most N-terminal repeat unit). In particular embodiments, the NTD polypeptide comprises at least about 121, 122, 123, 124, 125, 126, 127, 128, 129, 130, 131, 132, 133, 134, 135, 136, or 137 amino acids N-terminal to the TALE DNA binding domain of a Ralstonia TALE protein.

As used herein, the term “C-terminal domain (CTD)” polypeptide refers to the sequence that flanks the C-terminal portion or fragment of a naturally occurring TALE DNA binding domain. The CTD sequence, if present, may be of any length as long as the TALE DNA binding domain repeat units retain the ability to bind DNA. In particular embodiments, the CTD polypeptide comprises at least 20 to at least 85 or more amino acids C-terminal to the last full repeat of the TALE DNA binding domain (the first 20 amino acids are the half-repeat unit C-terminal to the last C-terminal full repeat unit). In particular embodiments, the CTD polypeptide comprises at least about 20, 21, 22, 23, 24, 25, 26, 27, 28, 29, 30, 31, 32, 33, 34, 35, 36, 37, 38, 39, 40, 41, 42, 443, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or at least 85 amino acids C-terminal to the last full repeat of the TALE DNA binding domain. In one embodiment, a megaTAL contemplated herein comprises a CTD polypeptide of at least about amino acids −20 to −1 of a Xanthomonas TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA binding domain of a Xanthomonas TALE protein. In one embodiment, a megaTAL contemplated herein comprises a CTD polypeptide of at least about amino acids −20 to −1 of a Ralstonia TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA binding domain of a Ralstonia TALE protein.

In particular embodiments, a megaTAL contemplated herein, comprises a fusion polypeptide comprising a TALE DNA binding domain engineered to bind a target sequence, a homing endonuclease reprogrammed to bind and cleave a target sequence and engineered to increase enzyme stability and/or activity, and optionally an NTD and/or CTD polypeptide, optionally joined to each other with one or more linker polypeptides contemplated elsewhere herein. Without wishing to be bound by any particular theory, it is contemplated that a megaTAL comprising TALE DNA binding domain, and optionally an NTD and/or CTD polypeptide is fused to a linker polypeptide which is further fused to a homing endonuclease variant. Thus, the TALE DNA binding domain binds a DNA target sequence that is within about 1, 2, 3, 4, 5, 6, 7, 8, 9, 10, 11, 12, 13, 14, or 15 nucleotides away from the target sequence bound by the DNA binding domain of the homing endonuclease variant. In this way, the megaTALs contemplated herein, increase the specificity and efficiency of genome editing.

In one embodiment, a megaTAL comprises a homing endonuclease variant and a TALE DNA binding domain that binds a nucleotide sequence that is within about 2, 3, 4, 5, or 6 nucleotides upstream of the binding site of the reprogrammed homing endonuclease.

In particular embodiments, a megaTAL contemplated herein, comprises one or more TALE DNA binding repeat units and an I-OnuI HE variant comprising increased thermostability and/or enzymatic activity compared to a parent I-OnuI HE variant.

In particular embodiments, a megaTAL contemplated herein, comprises an NTD, one or more TALE DNA binding repeat units, a CTD, and an I-OnuI HE variant comprising increased thermostability and/or enzymatic activity compared to a parent I-OnuI HE variant.

In particular embodiments, a megaTAL contemplated herein, comprises an NTD, about 9.5 to about 15.5 TALE DNA binding repeat units, and an I-OnuI HE variant comprising increased thermostability and/or enzymatic activity compared to a parent I-OnuI HE variant.

In particular embodiments, a megaTAL contemplated herein, comprises an NTD of about 122 amino acids to 137 amino acids, about 9.5, about 10.5, about 11.5, about 12.5, about 13.5, about 14.5, or about 15.5 binding repeat units, a CTD of about 20 amino acids to about 85 amino acids, and an I-OnuI HE variant comprising increased thermostability and/or enzymatic activity compared to a parent I-OnuI HE variant. In particular embodiments, any one of, two of, or all of the NTD, DNA binding domain, and CTD can be designed from the same species or different species, in any suitable combination.

In particular embodiments, a megaTAL comprising an I-OnuI HE variant that has one or more mutations to enhance thermostability is reprogrammed to bind a target site or sequence in a gene selected from the group consisting of: HBA, HBB, HBG1, HBG2, BCL11A, PCSK9, TCRA, TCRB, B2M, HLA-A, HLA-B, HLA-C, HLA-E, HLA-F, HLA-G, CIITA, AHR, PD-1, CTLA4, TIGIT, TGFBR2, LAG-3, TIM-3, BTLA, IL4R, IL6R, CXCR1, CXCR2, IL10R, IL13Ra2, TRAILR1, RCAS1R, and FAS.

### 3. End-Processing Enzymes

Genome editing compositions and methods contemplated in particular embodiments comprise editing cellular genomes using an I-OnuI HE variant comprising increased thermostability and/or enzymatic activity compared to a parent I-OnuI HE variant and one or more copies of an end-processing enzyme. In particular embodiments, a single polynucleotide encodes a homing endonuclease variant and an end-processing enzyme, separated by a linker, a self-cleaving peptide sequence, e.g., 2A sequence, or by an IRES sequence. In particular embodiments, genome editing compositions comprise a polynucleotide encoding a nuclease variant and a separate polynucleotide encoding an end-processing enzyme. In particular embodiments, genome editing compositions comprise a polynucleotide encoding a homing endonuclease variant end-processing enzyme single polypeptide fusion in addition to a tandem copy of the end-processing enzyme separated by a self-cleaving peptide.

The term “end-processing enzyme” refers to an enzyme that modifies the exposed ends of a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An end-processing enzyme may modify exposed polynucleotide chain ends by adding one or more nucleotides, removing one or more nucleotides, removing or modifying a phosphate group and/or removing or modifying a hydroxyl group. An end-processing enzyme may modify ends at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolysis and chemotherapy agents.

In particular embodiments, genome editing compositions and methods contemplated in particular embodiments comprise editing cellular genomes using and an I-OnuI HE variant comprising increased thermostability and/or enzymatic activity compared to a parent I-OnuI HE variant or megaTAL and a DNA end-processing enzyme.

The term “DNA end-processing enzyme” refers to an enzyme that modifies the exposed ends of DNA. A DNA end-processing enzyme may modify blunt ends or staggered ends (ends with 5′ or 3′ overhangs). A DNA end-processing enzyme may modify single stranded or double stranded DNA. A DNA end-processing enzyme may modify ends at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolysis and chemotherapy agents. DNA end-processing enzyme may modify exposed DNA ends by adding one or more nucleotides, removing one or more nucleotides, removing or modifying a phosphate group and/or removing or modifying a hydroxyl group.

Illustrative examples of DNA end-processing enzymes suitable for use in particular embodiments contemplated herein include but are not limited to: 5′-3′ exonucleases, 5′-3′ alkaline exonucleases, 3′-5′ exonucleases, 5′ flap endonucleases, helicases, phosphatases, hydrolases and template-independent DNA polymerases.

Additional illustrative examples of DNA end-processing enzymes suitable for use in particular embodiments contemplated herein include, but are not limited to, Trex2, Trex1, Trex1 without transmembrane domain, Apollo, Artemis, DNA2, Exo1, ExoT, ExoIII, Fen1, Fan1, MreII, Rad2, Rad9, TdT (terminal deoxynucleotidyl transferase), PNKP, RecE, RecJ, RecQ, Lambda exonuclease, Sox, Vaccinia DNA polymerase, exonuclease I, exonuclease III, exonuclease VII, NDK1, NDK5, NDK7, NDK8, WRN, T7-exonuclease Gene 6, avian myeloblastosis virus integration protein (IN), Bloom, Antartic Phophatase, Alkaline Phosphatase, Poly nucleotide Kinase (PNK), ApeI, Mung Bean nuclease, Hex1, TTRAP (TDP2), Sgs1, Sae2, CUP, Pol mu, Pol lambda, MUS81, EME1, EME2, SLX1, SLX4 and UL-12.

In particular embodiments, genome editing compositions and methods for editing cellular genomes contemplated herein comprise polypeptides comprising an I-OnuI HE variant or megaTAL and an exonuclease. The term “exonuclease” refers to enzymes that cleave phosphodiester bonds at the end of a polynucleotide chain via a hydrolyzing reaction that breaks phosphodiester bonds at either the 3′ or 5′ end.

Illustrative examples of exonucleases suitable for use in particular embodiments contemplated herein include, but are not limited to: hExoI, Yeast ExoI, E. coli ExoI, hTREX2, mouse TREX2, rat TREX2, hTREX1, mouse TREX1, rat TREX1, and Rat TREX1.

In particular embodiments, the DNA end-processing enzyme is a 3′ to 5′ exonuclease, preferably Trex 1 or Trex2, more preferably Trex2, and even more preferably human or mouse Trex2.

### D. Polypeptides

Various polypeptides are contemplated herein, including, but not limited to, homing endonuclease variants and megaTALs engineered to increase thermostability and/or enzymatic activity, and fusion polypeptides. In preferred embodiments, a polypeptide comprises the amino acid sequence set forth in any one or SEQ ID NOs: 9-14, 16-18, 22, and 23. “Polypeptide,” “peptide” and “protein” are used interchangeably, unless specified to the contrary, and according to conventional meaning, i.e., as a sequence of amino acids. In one embodiment, a “polypeptide” includes fusion polypeptides and other variants. Polypeptides can be prepared using any of a variety of well-known recombinant and/or synthetic techniques. Polypeptides are not limited to a specific length, e.g., they may comprise a full-length protein sequence, a fragment of a full-length protein, or a fusion protein, and may include post-translational modifications of the polypeptide, for example, glycosylations, acetylations, phosphorylations and the like, as well as other modifications known in the art, both naturally occurring and non-naturally occurring.

An “isolated protein,” “isolated peptide,” or “isolated polypeptide” and the like, as used herein, refer to in vitro synthesis, isolation, and/or purification of a peptide or polypeptide molecule from a cellular environment, and from association with other components of the cell, i.e., it is not significantly associated with in vivo substances. In particular embodiments, an isolated polypeptide is a synthetic polypeptide, a semi-synthetic polypeptide, or a polypeptide obtained or derived from a recombinant source.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more amino acids of the above polypeptide sequences. For example, in particular embodiments, it may be desirable to improve the biological properties of a homing endonuclease, megaTAL or the like that binds and cleaves a target site by introducing one or more substitutions, deletions, additions and/or insertions into the polypeptide that increase thermal stability. In particular embodiments, polypeptides include polypeptides having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity to a reference sequence, typically where the variant maintains at least one biological activity of the reference sequence.

In preferred embodiments, polypeptide variants include homing endonucleases or megaTALs that have been engineered to increase their thermostability and/or activity. I-OnuI HE polypeptides or fragments thereof can be reprogrammed to bind and cleave a target site. In particular embodiments, a reprogrammed I-OnuI HE variant has relatively low thermostability and/or activity compared to a parent I-OnuI HE. In preferred embodiments, an I-OnuI homing endonuclease or fragment thereof is engineered to bind and cleave a target site and to increase thermostability and/or activity of the enzyme.

Polypeptide variants include biologically active “polypeptide fragments.” Illustrative examples of biologically active polypeptide fragments include DNA binding domains, nuclease domains, and the like. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In preferred embodiments, the biological activity is binding affinity and/or cleavage activity for a target sequence. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long. In particular embodiments, a polypeptide comprises a biologically active fragment of a homing endonuclease variant. In particular embodiments, a polypeptide comprises a biologically active fragment of a homing endonuclease variant or a megaTAL. In particular embodiments, the polypeptides set forth herein may comprise one or more amino acids denoted as “X.” “X” if present in an amino acid SEQ ID NO, refers to any amino acid. One or more “X” residues may be present at the N- and C-terminus of an amino acid sequence set forth in particular SEQ ID NOs contemplated herein. If the “X” amino acids are not present the remaining amino acid sequence set forth in a SEQ ID NO may be considered a biologically active fragment.

A biologically active fragment may comprise an N-terminal truncation and/or C-terminal truncation. In a particular embodiment, a biologically active fragment lacks or comprises a deletion of the 1, 2, 3, 4, 5, 6, 7, or 8 N-terminal amino acids of a homing endonuclease variant compared to a corresponding wild type homing endonuclease sequence, more preferably a deletion of the 4 N-terminal amino acids of a homing endonuclease variant compared to a corresponding wild type homing endonuclease sequence. In a particular embodiment, a biologically active fragment lacks or comprises a deletion of the 1, 2, 3, 4, or 5 C-terminal amino acids of a homing endonuclease variant compared to a corresponding wild type homing endonuclease sequence, more preferably a deletion of the 2 C-terminal amino acids of a homing endonuclease variant compared to a corresponding wild type homing endonuclease sequence. In a particular preferred embodiment, a biologically active fragment lacks or comprises a deletion of the 4 N-terminal amino acids and 2 C-terminal amino acids of a homing endonuclease variant compared to a corresponding wild type homing endonuclease sequence.

In a particular embodiment, an I-OnuI variant comprises a deletion of 1, 2, 3, 4, 5, 6, 7, or 8 the following N-terminal amino acids: M, A, Y, M, S, R, R, E; and/or a deletion of the following 1, 2, 3, 4, or 5 C-terminal amino acids: R, G, S, F, V.

In a particular embodiment, an I-OnuI variant comprises a deletion or substitution of 1, 2, 3, 4, 5, 6, 7, or 8 the following N-terminal amino acids: M, A, Y, M, S, R, R, E; and/or a deletion or substitution of the following 1, 2, 3, 4, or 5 C-terminal amino acids: R, G, S, F, V.

In a particular embodiment, an I-OnuI variant comprises a deletion of 1, 2, 3, 4, 5, 6, 7, or 8 the following N-terminal amino acids: M, A, Y, M, S, R, R, E; and/or a deletion of the following 1 or 2 C-terminal amino acids: F, V.

In a particular embodiment, an I-OnuI variant comprises a deletion or substitution of 1, 2, 3, 4, 5, 6, 7, or 8 the following N-terminal amino acids: M, A, Y, M, S, R, R, E; and/or a deletion or substitution of the following 1 or 2 C-terminal amino acids: F, V.

As noted above, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82:488-492), Kunkel et al., (1987, Methods in Enzymol, 154:367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments, polypeptides include polypeptides having at least about and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 1.

TABLE 1
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |

| | | | | | | |
|---|---|---|---|---|---|---|
| Threonine | T | Thr | ACA | ACC | ACG | ACU |
| Valine | V | Val | GUA | GUC | GUG | GUU |

| Tryptophan | W | Trp | UGG | |
|---|---|---|---|---|
| Tyrosine | Y | Tyr | UAC | UAU |

Guidance in determining which amino acid residues can be substituted, inserted, or deleted in particular embodiments, without abolishing biological activity can be found using computer programs well known in the art, such as DNASTAR, DNA Strider, Geneious, Mac Vector, or Vector NTI software. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

In one embodiment, an I-OnuI variant comprises one or more non-conservative amino acid substitutions at positions that affect the thermostability of the enzyme. In one embodiment, an I-OnuI variant comprises one or more conservative and/or non-conservative amino acid substitutions at positions that affect the thermostability of the enzyme.

In particular embodiments, where expression of two or more polypeptides is desired, the polynucleotide sequences encoding them can be separated by and IRES sequence as disclosed elsewhere herein.

Polypeptides contemplated in particular embodiments include fusion polypeptides. In particular embodiments, fusion polypeptides and polynucleotides encoding fusion polypeptides are provided. Fusion polypeptides and fusion proteins refer to a polypeptide having at least two, three, four, five, six, seven, eight, nine, or ten polypeptide segments.

In another embodiment, two or more polypeptides can be expressed as a fusion protein that comprises one or more self-cleaving polypeptide sequences as disclosed elsewhere herein.

In one embodiment, a fusion protein contemplated herein comprises one or more DNA binding domains and one or more nucleases, and one or more linker and/or self-cleaving polypeptides.

In one embodiment, a fusion protein contemplated herein comprises nuclease variant; a linker or self-cleaving peptide; and an end-processing enzyme including but not limited to a 5′-3′ exonuclease, a 5′-3′ alkaline exonuclease, and a 3′-5′ exonuclease (e.g., Trex2).

Fusion polypeptides can comprise one or more polypeptide domains or segments including, but are not limited to signal peptides, cell permeable peptide domains (CPP), DNA binding domains, nuclease domains, etc., epitope tags (e.g., maltose binding protein (“MBP”), glutathione S transferase (GST), HIS6, MYC, FLAG, V5, VSV-G, and HA), polypeptide linkers, and polypeptide cleavage signals. Fusion polypeptides are typically linked C-terminus to N-terminus, although they can also be linked C-terminus to C-terminus, N-terminus to N-terminus, or N-terminus to C-terminus. In particular embodiments, the polypeptides of the fusion protein can be in any order. Fusion polypeptides or fusion proteins can also include conservatively modified variants, polymorphic variants, alleles, mutants, subsequences, and interspecies homologs, so long as the desired activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as disclosed elsewhere herein.

Fusion polypeptides may optionally comprise a linker that can be used to link the one or more polypeptides or domains within a polypeptide. A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. Linker sequences are not required when a particular fusion polypeptide segment contains non-essential N-terminal amino acid regions that can be used to separate the functional domains and prevent steric interference. Preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary linkers include, but are not limited to the following amino acid sequences: glycine polymers (G)ₙ; glycine-serine polymers (G₁₋₅S₁₋₅)ₙ, where n is an integer of at least one, two, three, four, or five; glycine-alanine polymers; alanine-serine polymers; GGG (SEQ ID NO: 24); DGGGS (SEQ ID NO: 25); TGEKP (SEQ ID NO: 26) (see e.g., Liu et al., PNAS 5525-5530 (1997)); GGRR (SEQ ID NO: 27) (Pomerantz et al. 1995, supra); (GGGGS)ₙ wherein n=1, 2, 3, 4 or 5 (SEQ ID NO: 28) (Kim et al., PNAS 93, 1156-1160 (1996.); EGKSSGSGSESKVD (SEQ ID NO: 29) (Chaudhary et al., 1990, Proc. Natl. Acad. Sci. U.S.A. 87:1066-1070); KESGSVSSEQLAQFRSLD (SEQ ID NO: 30) (Bird et al., 1988, Science 242:423-426), GGRRGGGS (SEQ ID NO: 31); LRQRDGERP (SEQ ID NO: 32); LRQKDGGGSERP (SEQ ID NO: 33); LRQKD (GGGS)₂ERP (SEQ ID NO: 34). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

Fusion polypeptides may further comprise a polypeptide cleavage signal between each of the polypeptide domains described herein or between an endogenous open reading frame and a polypeptide encoded by a donor repair template. In addition, a polypeptide cleavage site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5 (8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ (G/S) (SEQ ID NO: 35), for example, ENLYFQG (SEQ ID NO: 36) and ENLYFQS (SEQ ID NO: 37), wherein X represents any amino acid (cleavage by TEV occurs between Q and G or Q and S).

In particular embodiments, the polypeptide cleavage signal is a viral self-cleaving peptide or ribosomal skipping sequence.

Illustrative examples of ribosomal skipping sequences include but are not limited to: a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of 2A sites are provided in Table 2.

TABLE 2
Exemplary 2A sites include the following
sequences:

| | |
|---|---|
| SEQ ID NO: 38 | GSGATNFSLLKQAGDVEENPGP |
| SEQ ID NO: 39 | ATNFSLLKQAGDVEENPGP |
| SEQ ID NO: 40 | LLKQAGDVEENPGP |
| SEQ ID NO: 41 | GSGEGRGSLLTCGDVEENPGP |
| SEQ ID NO: 42 | EGRGSLLTCGDVEENPGP |
| SEQ ID NO: 43 | LLTCGDVEENPGP |
| SEQ ID NO: 44 | GSGQCTNYALLKLAGDVESNPGP |
| SEQ ID NO: 45 | QCTNYALLKLAGDVESNPGP |
| SEQ ID NO: 46 | LLKLAGDVESNPGP |
| SEQ ID NO: 47 | GSGVKQTLNFDLLKLAGDVESNPGP |
| SEQ ID NO: 48 | VKQTLNFDLLKLAGDVESNPGP |
| SEQ ID NO: 49 | LLKLAGDVESNPGP |
| SEQ ID NO: 50 | LLNFDLLKLAGDVESNPGP |
| SEQ ID NO: 51 | TLNFDLLKLAGDVESNPGP |
| SEQ ID NO: 52 | LLKLAGDVESNPGP |
| SEQ ID NO: 53 | NFDLLKLAGDVESNPGP |
| SEQ ID NO: 54 | QLLNFDLLKLAGDVESNPGP |
| SEQ ID NO: 55 | APVKQTLNFDLLKLAGDVESNPGP |
| SEQ ID NO: 56 | VTELLYRMKRAETYCPRPLLAIHPTEARHKQKI |
| | VAPVKQT |
| SEQ ID NO: 57 | LNFDLLKLAGDVESNPGP |
| SEQ ID NO: 58 | LLAIHPTEARHKQKIVAPVKQTLNFDLLKLAGD |
| | VESNPGP |
| SEQ ID NO: 59 | EARHKQKIVAPVKQTLNFDLLKLAGDVESNPGP |

### E. Polynucleotides

In particular embodiments, polynucleotides encoding one or more homing endonuclease variants and megaTALs engineered to increase thermostability and/or enzymatic activity, and fusion polypeptides contemplated herein are provided. As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, genomic RNA (gRNA), plus strand RNA (RNA (+)), minus strand RNA (RNA (−)), tracrRNA, crRNA, single guide RNA (sgRNA), synthetic RNA, synthetic mRNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

As used herein the term “nucleotide” refers to a heterocyclic nitrogenous base in N-glycosidic linkage with a phosphorylated sugar. Nucleotides are understood to include natural bases, and a wide variety of art-recognized modified bases. Such bases are generally located at the 1′ position of a nucleotide sugar moiety. Nucleotides generally comprise a base, sugar and a phosphate group. In ribonucleic acid (RNA), the sugar is a ribose, and in deoxyribonucleic acid (DNA) the sugar is a deoxyribose, i.e., a sugar lacking a hydroxyl group that is present in ribose. Exemplary natural nitrogenous bases include the purines, adenosine (A) and guanidine (G), and the pyrimidines, cytidine (C) and thymidine (T) (or in the context of RNA, uracil (U)). The C-1 atom of deoxyribose is bonded to N-1 of a pyrimidine or N-9 of a purine. Nucleotides are usually mono, di- or triphosphates. The nucleotides can be unmodified or modified at the sugar, phosphate and/or base moiety, (also referred to interchangeably as nucleotide analogs, nucleotide derivatives, modified nucleotides, non-natural nucleotides, and non-standard nucleotides; see for example, WO 92/07065 and WO 93/15187). Examples of modified nucleic acid bases are summarized by Limbach et al., (1994, Nucleic Acids Res. 22, 2183-2196).

A nucleotide may also be regarded as a phosphate ester of a nucleoside, with esterification occurring on the hydroxyl group attached to C-5 of the sugar. As used herein, the term “nucleoside” refers to a heterocyclic nitrogenous base in N-glycosidic linkage with a sugar. Nucleosides are recognized in the art to include natural bases, and also to include well known modified bases. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, nucleoside derivatives, modified nucleosides, non-natural nucleosides, or non-standard nucleosides). As also noted above, examples of modified nucleic acid bases are summarized by Limbach et al., (1994, Nucleic Acids Res. 22, 2183-2196).

Illustrative examples of polynucleotides include, but are not limited to polynucleotides encoding SEQ ID NOs: 9-14, 16-18, 22, and 23 and polynucleotide sequences set forth in SEQ ID NOs: 19-21.

In various illustrative embodiments, polynucleotides contemplated herein include, but are not limited to polynucleotides encoding homing endonuclease variants, megaTALs, end-processing enzymes, fusion polypeptides, and expression vectors, viral vectors, and transfer plasmids comprising polynucleotides contemplated herein.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

In one embodiment, a polynucleotide comprises a nucleotide sequence that hybridizes to a target nucleic acid sequence under stringent conditions. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc., 1994-1998, Chapter 15.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant polynucleotide, a synthetic polynucleotide, or other polynucleotide that does not exist in nature and that has been made by the hand of man. In particular embodiments, an isolated polynucleotide is a synthetic polynucleotide, a semi-synthetic polynucleotide, or a polynucleotide obtained or derived from a recombinant source.

In various embodiments, a polynucleotide comprises an mRNA encoding a polypeptide contemplated herein including, but not limited to, a homing endonuclease variant, a megaTAL, and an end-processing enzyme. In certain embodiments, the mRNA comprises a cap, one or more nucleotides, and a poly(A) tail.

As used herein, the terms “5′ cap” or “5′ cap structure” or “5′ cap moiety” refer to a chemical modification, which has been incorporated at the 5′ end of an mRNA. The 5′ cap is involved in nuclear export, mRNA stability, and translation.

In particular embodiments, a mRNA encoding a homing endonuclease variant or megaTAL comprises a 5′ cap comprising a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue.

Illustrative examples of 5′ cap suitable for use in particular embodiments of the mRNA polynucleotides contemplated herein include, but are not limited to: unmethylated 5′ cap analogs, e.g., G(5′)ppp(5)G, G(52)ppp(5″)C, G(5′)ppp(5′)A; methylated 5′ cap analogs, e.g., m⁷G(5′)ppp(5′)G, m⁷G(5′)ppp(5′)C, and m⁷G(5′)ppp(5′)A; dimethylated 5′ cap analogs, e.g., m^{2,7}G(5″)ppp(5′)G, m^{2,7}G(5′)ppp(5′)C, and m^{2,7}G(5′)ppp(5′)A; trimethylated 5′ cap analogs, e.g., m^{2.2,7}G(5′)ppp(5″)G, m^{2,2,7}G(5′)ppp(5′)C, and m^{2,2,7}G(5′)ppp(5′)A; dimethylated symmetrical 5′ cap analogs, e.g., m⁷G(5′)pppm⁷(5′)G, m⁷G(5′)pppm⁷(5′)C, and m⁷G(5′)pppm⁷(5′)A; and anti-reverse 5′ cap analogs, e.g., Anti-Reverse Cap Analog (ARCA) cap, designated 3′O-Me-m⁷G(5′)ppp(5′)G, 2′O-Me-m⁷G(5′)ppp(5′)G, 2′O-Me-m⁷G(5)ppp(5′)C, 2′O-Me-m⁷G(5)ppp(5′)A, m⁷²′d(5)ppp(5′)G, m⁷²′d(52)ppp(5′)C, m⁷²′d(5)ppp(5′)A, 3′O-Me-m⁷G(5′)ppp(5′)C, 3′O-Me-m⁷G(5′)ppp(5′)A, m⁷³′d(5)ppp(5″)G, m⁷³′d(5′)ppp(5′)C, m⁷³′d(5′)ppp(5′)A and their tetraphosphate derivatives) (see, e.g., Jemielity et al., RNA, 9:1108-1122 (2003)).

In particular embodiments, mRNAs encoding a homing endonuclease variant or megaTAL comprise a 5′ cap that is a 7-methyl guanylate (“m⁷G”) linked via a triphosphate bridge to the 5′-end of the first transcribed nucleotide, resulting in m⁷G(5′)ppp(5) N, where N is any nucleoside.

In some embodiments, mRNAs encoding a homing endonuclease variant or megaTAL comprise a 5′ cap wherein the cap is a Cap0 structure (Cap0 structures lack a 2′-O-methyl residue of the ribose attached to bases 1 and 2), a Cap1 structure (Cap1 structures have a 2′-O-methyl residue at base 2), or a Cap2 structure (Cap2 structures have a 2′-O-methyl residue attached to both bases 2 and 3).

In one embodiment, an mRNA comprises a m⁷G(5′)ppp(5)G cap.

In one embodiment, an mRNA comprises an ARCA cap.

In particular embodiments, an mRNA encoding a homing endonuclease variant or megaTAL comprises one or more modified nucleosides.

In one embodiment, an mRNA encoding a homing endonuclease variant or megaTAL comprises one or more modified nucleosides selected from the group consisting of: pseudouridine, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In one embodiment, an mRNA encoding a homing endonuclease variant or megaTAL comprises one or more modified nucleosides selected from the group consisting of: pseudouridine, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In one embodiment, an mRNA encoding a homing endonuclease variant or megaTAL comprises one or more modified nucleosides selected from the group consisting of: 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In one embodiment, an mRNA encoding a homing endonuclease variant or megaTAL comprises one or more modified nucleosides selected from the group consisting of: 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In one embodiment, an mRNA encoding a homing endonuclease variant or megaTAL comprises one or more modified nucleosides selected from the group consisting of: inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In one embodiment, an mRNA comprises one or more pseudouridines, one or more 5-methyl-cytosines, and/or one or more 5-methyl-cytidines.

In one embodiment, an mRNA comprises one or more pseudouridines.

In one embodiment, an mRNA comprises one or more 5-methyl-cytidines.

In one embodiment, an mRNA comprises one or more 5-methyl-cytosines.

In particular embodiments, an mRNA encoding a homing endonuclease variant or megaTAL comprises a poly(A) tail to help protect the mRNA from exonuclease degradation, stabilize the mRNA, and facilitate translation. In certain embodiments, an mRNA comprises a 3′ poly(A) tail structure.

In particular embodiments, the length of the poly(A) tail is at least about 10, 25, 50, 75, 100, 150, 200, 250, 300, 350, 400, 450, or at least about 500 or more adenine nucleotides or any intervening number of adenine nucleotides. In particular embodiments, the length of the poly(A) tail is at least about 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, or 275 or more adenine nucleotides.

In particular embodiments, the length of the poly(A) tail is about 10 to about 500 adenine nucleotides, about 50 to about 500 adenine nucleotides, about 100 to about 500 adenine nucleotides, about 150 to about 500 adenine nucleotides, about 200 to about 500 adenine nucleotides, about 250 to about 500 adenine nucleotides, about 300 to about 500 adenine nucleotides, about 50 to about 450 adenine nucleotides, about 50 to about 400 adenine nucleotides, about 50 to about 350 adenine nucleotides, about 100 to about 500 adenine nucleotides, about 100 to about 450 adenine nucleotides, about 100 to about 400 adenine nucleotides, about 100 to about 350 adenine nucleotides, about 100 to about 300 adenine nucleotides, about 150 to about 500 adenine nucleotides, about 150 to about 450 adenine nucleotides, about 150 to about 400 adenine nucleotides, about 150 to about 350 adenine nucleotides, about 150 to about 300 adenine nucleotides, about 150 to about 250 adenine nucleotides, about 150 to about 200 adenine nucleotides, about 200 to about 500 adenine nucleotides, about 200 to about 450 adenine nucleotides, about 200 to about 400 adenine nucleotides, about 200 to about 350 adenine nucleotides, about 200 to about 300 adenine nucleotides, about 250 to about 500 adenine nucleotides, about 250 to about 450 adenine nucleotides, about 250 to about 400 adenine nucleotides, about 250 to about 350 adenine nucleotides, or about 250 to about 300 adenine nucleotides or any intervening range of adenine nucleotides.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the pre-messenger (pre-mRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The polynucleotides contemplated in particular embodiments, regardless of the length of the coding sequence itself, may be combined with other DNA sequences, such as promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, post-transcription response elements, and polynucleotides encoding self-cleaving polypeptides, epitope tags, as disclosed elsewhere herein or as known in the art, such that their overall length may vary considerably. It is therefore contemplated in particular embodiments that a polynucleotide fragment of almost any length may be employed, with the total length preferably being limited by the ease of preparation and use in the intended recombinant DNA protocol.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector. A desired polypeptide can also be expressed by delivering an mRNA encoding the polypeptide into the cell.

Illustrative examples of vectors include, but are not limited to plasmid, autonomously replicating sequences, and transposable elements, e.g., Sleeping Beauty, PiggyBac.

Additional illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

“Expression control sequences,” “control elements,” or “regulatory sequences” present in an expression vector are those non-translated regions of the vector including but not limited to an origin of replication, selection cassettes, promoters, enhancers, translation initiation signals (Shine Dalgarno sequence or Kozak sequence) introns, post-transcriptional regulatory elements, a polyadenylation sequence, 5′ and 3′ untranslated regions, which interact with host cellular proteins to carry out transcription and translation. Such elements may vary in their strength and specificity. Depending on the vector system and host utilized, any number of suitable transcription and translation elements, including ubiquitous promoters and inducible promoters may be used.

The term “operably linked” refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rβgpA), variants thereof, or another suitable heterologous or endogenous polyA sequence known in the art. In particular embodiments, the poly(A) sequence is synthetic.

In particular embodiments, polynucleotides encoding one or more nuclease variants, megaTALs, end-processing enzymes, or fusion polypeptides may be introduced into a cell by both non-viral and viral methods.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a T cell.

Illustrative examples of non-viral vectors include, but are not limited to plasmids (e.g., DNA plasmids or RNA plasmids), transposons, cosmids, and bacterial artificial chromosomes.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid: nucleic acid conjugates, naked DNA, artificial virions, DEAE-dextran-mediated transfer, gene gun, and heat-shock.

Illustrative examples of viral vector systems suitable for use in particular embodiments contemplated herein include, but are not limited to adeno-associated virus (AAV), retrovirus, e.g., lentivirus, herpes simplex virus, adenovirus, and vaccinia virus vectors.

### F. Compositions and Formulations

The compositions contemplated in particular embodiments may comprise one or more homing endonuclease variants and megaTALs engineered to increase thermostability and/or enzymatic activity, polynucleotides, vectors comprising same, and genome editing compositions and genome edited cell compositions, as contemplated herein. The genome editing compositions and methods contemplated in particular embodiments are useful for editing a target site in the human genome in a cell or a population of cells.

An “isolated cell” refers to a non-naturally occurring cell, e.g., a cell that does not exist in nature, a modified cell, an engineered cell, a recombinant cell etc., that has been obtained from an in vivo tissue or organ and is substantially free of extracellular matrix.

As used herein, the term “population of cells” refers to a plurality of cells that may be made up of any number and/or combination of homogenous or heterogeneous cell types.

In particular embodiments, a genome editing composition is used to edit a target site in an embryonic stem cell or an adult stem or progenitor cell.

In particular embodiments, a genome editing composition is used to edit a target site in a stem or progenitor cell selected from the group consisting of: mesodermal stem or progenitor cells, endodermal stem or progenitor cells, and ectodermal stem or progenitor cells. Illustrative examples of mesodermal stem or progenitor cells include but are not limited to bone marrow stem or progenitor cells, umbilical cord stem or progenitor cells, adipose tissue derived stem or progenitor cells, hematopoietic stem or progenitor cells (HSPCs), mesenchymal stem or progenitor cells, muscle stem or progenitor cells, kidney stem or progenitor cells, osteoblast stem or progenitor cells, chondrocyte stem or progenitor cells, and the like. Illustrative examples of ectodermal stem or progenitor cells include but are not limited to neural stem or progenitor cells, retinal stem or progenitor cells, skin stem or progenitor cells, and the like. Illustrative examples of endodermal stem or progenitor cells include but are not limited to liver stem or progenitor cells, pancreatic stem or progenitor cells, epithelial stem or progenitor cells, and the like.

In particular embodiments, a genome editing composition is used to edit a target site in a bone cell, osteocyte, osteoblast, adipose cell, chondrocyte, chondroblast, muscle cell, skeletal muscle cell, myoblast, myocyte, smooth muscle cell, bladder cell, bone marrow cell, central nervous system (CNS) cell, peripheral nervous system (PNS) cell, glial cell, astrocyte cell, neuron, pigment cell, epithelial cell, skin cell, endothelial cell, vascular endothelial cell, breast cell, colon cell, esophagus cell, gastrointestinal cell, stomach cell, colon cell, head cell, neck cell, gum cell, tongue cell, kidney cell, liver cell, lung cell, nasopharynx cell, ovary cell, follicular cell, cervical cell, vaginal cell, uterine cell, pancreatic cell, pancreatic parenchymal cell, pancreatic duct cell, pancreatic islet cell, prostate cell, penile cell, gonadal cell, testis cell, hematopoietic cell, lymphoid cell, or myeloid cell.

In a preferred embodiment, a genome editing composition is used to edit a target site in hematopoietic cells, e.g., hematopoietic stem cells, hematopoietic progenitor cells, CD34+ cells, immune effector cells, T cells, NKT cells, NK cells and the like.

In various embodiments, the compositions contemplated herein comprise I-OnuI HE variant engineered to increase thermostability and/or enzymatic activity, and optionally an end-processing enzyme, e.g., a 3′-5′ exonuclease (Trex2). The I-OnuI HE variant may be in the form of an mRNA that is introduced into a cell via polynucleotide delivery methods disclosed supra, e.g., electroporation, lipid nanoparticles, etc. In one embodiment, a composition comprising an mRNA encoding an I-OnuI HE variant or megaTAL, and optionally a 3′-5′ exonuclease, is introduced in a cell via polynucleotide delivery methods disclosed supra. The composition may be used to generate a genome edited cell or population of genome edited cells by error prone NHEJ.

In various embodiments, the compositions contemplated herein comprise a donor repair template. The composition may be delivered to a cell that expresses or will express an I-OnuI HE variant, and optionally an end-processing enzyme. In one embodiment, the composition may be delivered to a cell that expresses or will express an I-OnuI HE variant or megaTAL, and optionally a 3′-5′ exonuclease. Expression of the gene editing enzymes in the presence of the donor repair template can be used to generate a genome edited cell or population of genome edited cells by HDR.

In particular embodiments, a composition comprises a cell containing one or more homing endonuclease variants and megaTALs engineered to increase thermostability and/or enzymatic activity, polynucleotides, vectors comprising same. In particular embodiments, the cells may be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are obtained from a mammalian subject. In a more preferred embodiment, the cells are obtained from a primate subject, optionally a non-human primate. In the most preferred embodiment, the cells are obtained from a human subject.

In particular embodiments, the compositions contemplated herein comprise a population of cells, an I-OnuI HE variant, and optionally, a donor repair template. In particular embodiments, the compositions contemplated herein comprise a population of cells, an I-OnuI HE variant, an end-processing enzyme, and optionally, a donor repair template. The I-OnuI HE and/or end-processing enzyme may be in the form of an mRNA that is introduced into the cell via polynucleotide delivery methods disclosed supra.

In particular embodiments, the compositions contemplated herein comprise a population of cells, an I-OnuI HE variant or megaTAL engineered to increase thermostability and/or activity of the enzyme, and optionally, a donor repair template. In particular embodiments, the compositions contemplated herein comprise a population of cells, an I-OnuI HE variant or megaTAL, a 3′-5′ exonuclease, and optionally, a donor repair template. The I-OnuI HE variant, megaTAL, and/or 3′-5′ exonuclease may be in the form of an mRNA that is introduced into the cell via polynucleotide delivery methods disclosed supra.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Identification of Amino Acid Positions in a LADLIDADG Homing Endonuclease that Increase Thermostability

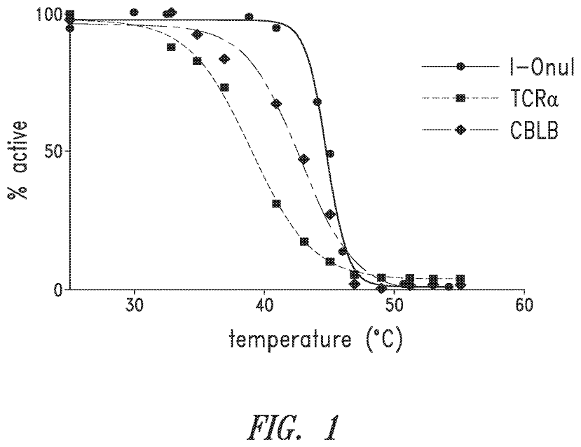

A yeast surface display assay was used to identify mutations that increase the thermostability of LAGLIDADG homing endonucleases. First, the stability of I-OnuI (e.g., SEQ ID NO: 1) and engineered nucleases (e.g., SEQ ID NOs: 6 and 7) was measured. After nuclease surface expression was induced in the yeast, each yeast population was subjected to heat shock at multiple temperatures for 15 minutes, and the percent of nuclease expressing cells still able to cleave its DNA target was measured by flow cytometry. This assay generates a standard protein melt curve with an associated TM₅₀ value for each endonuclease. FIG. 1.

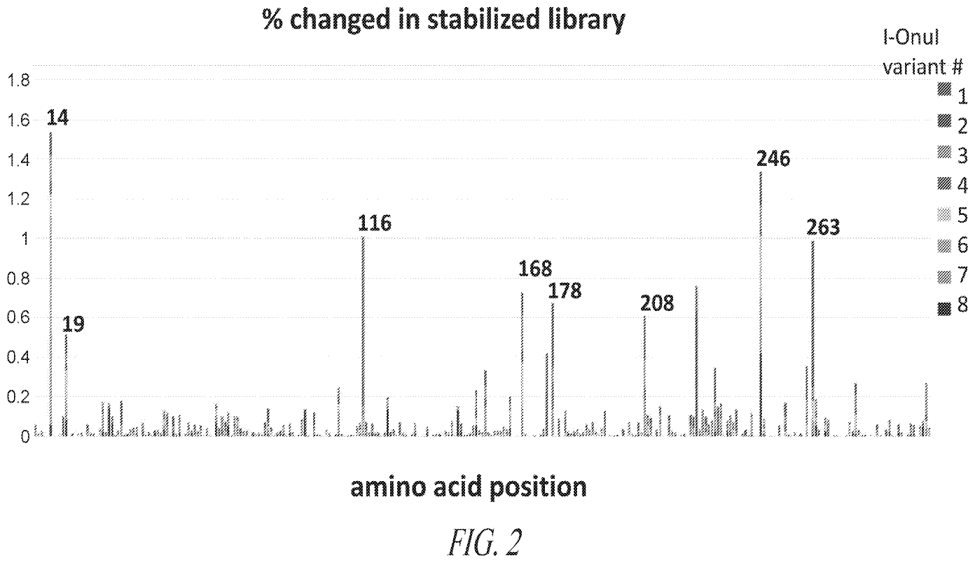

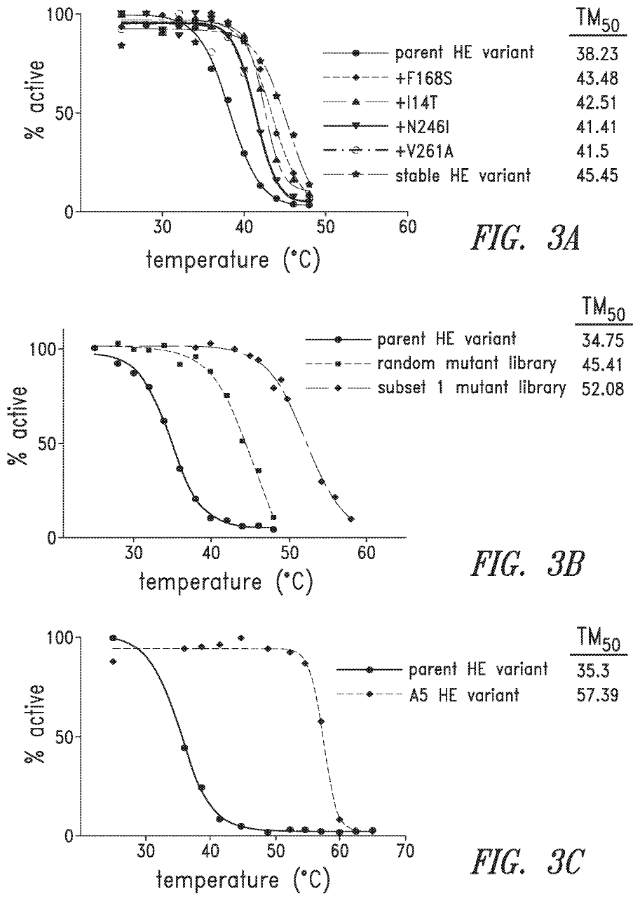

To identify mutations that lead to increased stability, multiple I-OnuI derived homing endonucleases were subjected to random mutagenesis via PCR over the entire open reading frame. These mutant libraries were expressed in yeast and sorted for active nuclease activity after heat shock at or above the TM₅₀ of the library. After two rounds of sorting, HE variants were sequenced with either PacBio or Sanger sequencing to determine the identity and frequency of mutations at each position. The cumulative mutation frequencies are shown in FIG. 2 as a stacked bar graph, and top mutations at each position are shown in Table 2. FIG. 3A shows the thermostability on a parent I-OnuI HE variant targeting BCL11A (SEQ ID NO 8), I-OnuI HE variants with single amino acid substitutions and their impact on thermostability (SEQ ID NOs: 9-12), and a representative I-OnuI HE variant (SEQ ID NO 13) generated from random mutagenesis that has a slightly higher overall TM₅₀ than the individual variants. FIG. 3A

### Example 2

### Combinatorial I-OnuI HE Stabilizing Mutations Increase Thermostability

To further increase I-OnuI HE variant thermostability, the most frequently mutated amino acid positions were combined in a single library. Starting with BCL11A I-OnuI HE variant (SEQ ID NO 8), residues 14, 153, 156, 168, 178, 208, 261 and 300 were mutated using degenerate codons and PCR (subset 1 mutant library). Sorting of this library at a relatively permissive temperature of 46° C. resulted in a population of variants that were 10° C. more stable than the products from a random mutant library (FIG. 3B). One representative BCL11A I-OnuI HE variant from the subset 1 mutant library (A5, SEQ ID NO 14) showed an unexpected 22° C. increase in thermostability compared to the parent I-OnuI HE variant (FIG. 3C). Combinatorial mutations derived from either random mutagenesis or directed mutagenesis increase I-Onu HE variant thermostability.

### Example 3

### Mutations that Increase Thermostability can be Transferred Between I-OnuI HE Variants

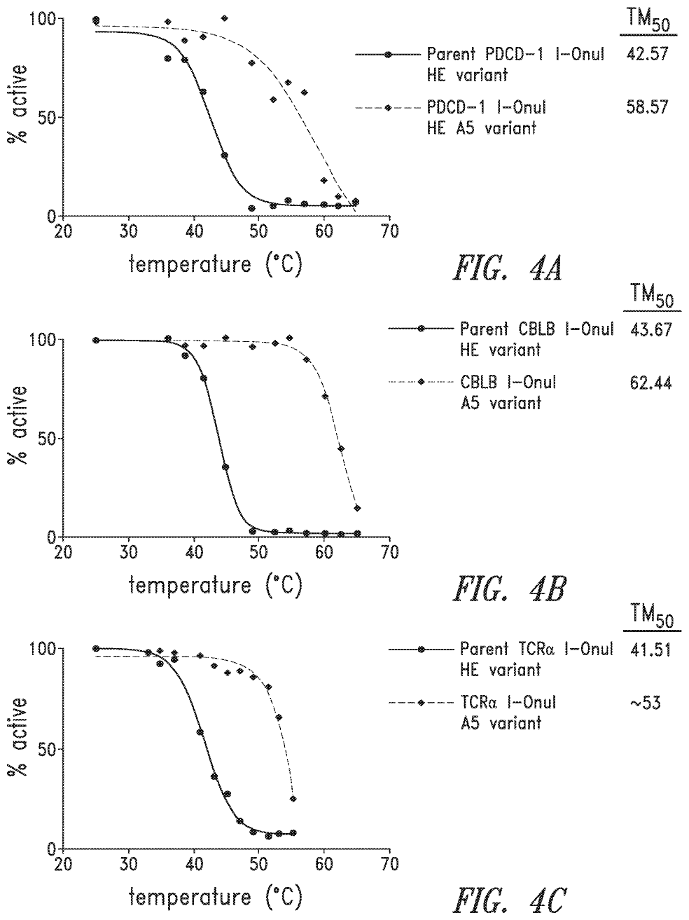

To determine if stabilizing mutations are unique to each reprogrammed I-OnuI HE variant or whether stabilizing mutations can be transferred between enzymes, the mutations from the BCL11A A5 I-OnuI HE variant (SEQ ID NO: 14) were transferred to I-OnuI HE variants that target PDCD-1 (SEQ ID NO: 15), TCRα (SEQ ID NO: 6), or CBLB (SEQ ID NO: 7). With these mutations, the TM₅₀ of an I-OnuI HE variant that targets the human PDCD-1 gene was increased by 16° C. (SEQ ID NO: 16), the TM₅₀ of an I-OnuI HE variant that targets the human TCRα gene was increased by ˜14° C. (SEQ ID NO: 17), and the TM₅₀ of an I-OnuI HE variant that targets the human CBLB gene was increased by 19° C. (SEQ ID NO 18). FIGS. 4A-4C. Mutations that increase thermostability were transferrable among different I-OnuI HE variants.

### Example 4

### Increased Thermostability Increases the Duration of I-OnuI HE Variant Expression

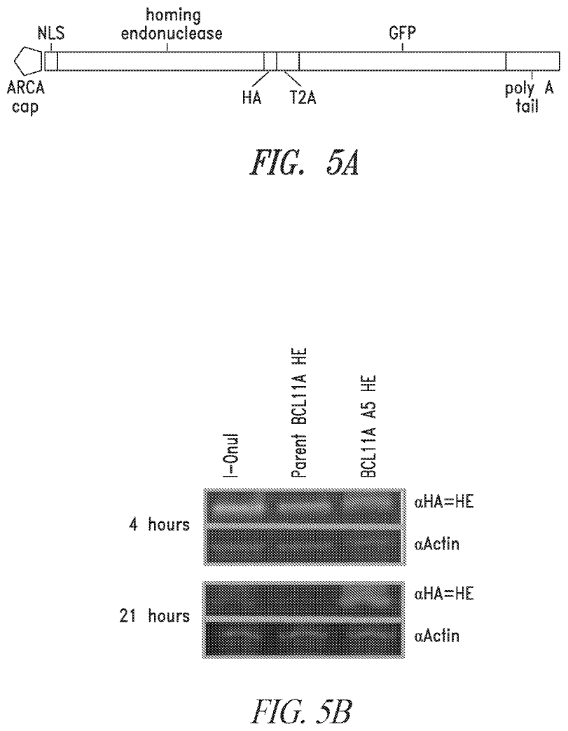

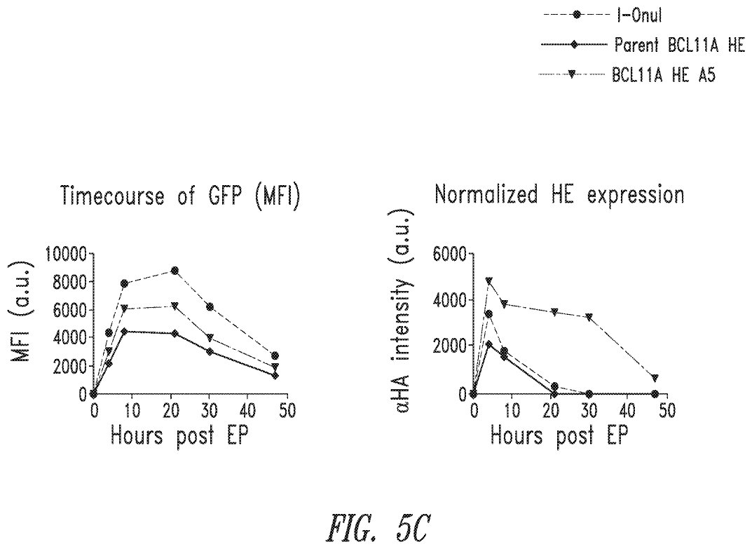

I-OnuI HE variant thermostability was also assessed by measuring expression of the enzymes in 293T cells. Briefly, each I-OnuI HE variant was formatted as an mRNA with a c-terminal HA tag followed by T2A GFP to track mRNA transfection efficiency (FIG. 5A). mRNA was prepared by in vitro transcription and co-transcriptionally capped with Anti-Reverse Cap Analog and enzymatically polyadenylated with poly(A) polymerase. The mRNA was purified and equal amounts electroporated into 293T cells (protein SEQ ID NOs 2,8,14: mRNA SEQ ID NOs: 19-21). At each time point cells were run on a cytometer to measure GFP expression, as well as lysed and frozen for western blot analysis.

The dynamics for GFP protein expression were similar for each polycistronic mRNA; but the amount of HA tagged HE protein varied (FIGS. 5B and 5C). Four hours after electroporation, the amount of stabilized BCL11A A5 HE protein was significant higher compared to the amount of parent BCL11A HE protein, when normalized to the actin loading control. Moreover, at the later time points, e.g., 21 hours, the amount of parent BCL11A HE protein was undetectable; in contrast, the BCL11A A5 HE variant was still near its peak expression levels. Overall, the stabilized BCL11A A5 HE variant was present in the cells for almost twice as long as the parent HE.

### Example 5

### I-OnuI HE Variant Engineered to Increase Thermostability Shows Increase Catalytic Activity

The effects of the stabilizing mutations on PDCD-1 editing was measured by comparing editing rates of a parental megaTAL that lacks the stabilizing mutations (SEQ ID NO: 22) with a megaTAL comprising stabilizing mutations (SEQ ID NO: 23). megaTAL mRNA was prepared by in vitro transcription, co-transcriptionally capped with Anti-Reverse Cap Analog (ARCA) and enzymatically polyadenylated with poly(A) polymerase. Purified mRNA was used to measure PDCD-1 editing efficiency in primary human T cells.

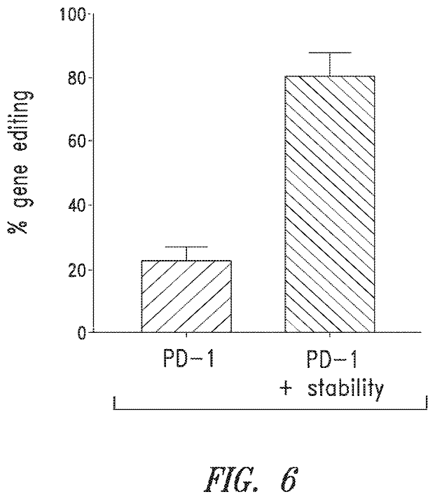

Primary human peripheral blood mononuclear cells (PBMCs) from two donors were activated with anti-CD3 and anti-CD28 antibodies and cultured in the presence of 250 U/mL IL-2. At 3 days post-activation cells were electroporated with megaTAL mRNA. Transfected T cells were expanded for an additional 7-10 days and editing efficiency was measured using sequencing across the PDCD-1 target site and Tracking of Indels by Decomposition (TIDE, see Brinkman et al., 2014) (FIG. 6). Without the stabilizing mutations, the PDCD-1 megaTAL showed low levels of editing (<20%); the stabilized PDCD-1 megaTAL increased editing activity to nearly 80%.

In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. An I-OnuI homing endonuclease (HE) variant comprising one or more amino acid substitutions relative to a parent I-OnuI HE comprising the amino acid sequence set forth in SEQ ID NO: 1, wherein the one or more amino acid substitutions increase the thermostability of the I-OnuI HE variant compared to the parent I-OnuI HE.

2. The I-OnuI HE variant of claim 1, wherein the one or more amino acid substitutions is at an amino acid position selected from the group consisting of: I14, A19, V116, F168, D208, N246, and L263.

3. The I-OnuI HE variant of claim 1, wherein the I-OnuI HE variant comprises amino acid substitutions at the following amino acid positions: I14, A19, F168, D208, and N246.

4. The I-OnuI HE variant of claim 1, wherein the one or more amino acid substitutions is at an amino acid position selected from the group consisting of: K108, K156, S176, E231, V261, E277, and G300.

5. The I-OnuI HE variant of claim 1, wherein the one or more amino acid substitutions is at an amino acid position selected from the group consisting of: N31, N33, K52, Y97, K124, K147, I153, K209, E264, and D268.

6. The I-OnuI HE variant of claim 1, wherein the I-OnuI HE variant comprises three or more amino acid substitutions is at an amino acid position selected from the group consisting of: I14, A19, K108, V116, K156, F168, S176, D208, E231, N246, V261, L263, E277, and G300.

7. The I-OnuI HE variant of claim 1, wherein the I-OnuI HE variant comprises three or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

8. The I-OnuI HE variant of claim 1, wherein the I-OnuI HE variant comprises five or more amino acid substitutions is at an amino acid position selected from the group consisting of: I14, A19, K108, V116, K156, F168, S176, D208, E231, N246, V261, L263, E277, and G300.

9. The I-OnuI HE variant of claim 1, wherein the I-OnuI HE variant comprises five or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

10. The I-OnuI HE variant of any one of claims 1 to 9, wherein the I-OnuI HE variant has a TM₅₀ at least 10° C. higher than the TM₅₀ of the parent I-OnuI HE.

11. The I-OnuI HE variant of any one of claims 1 to 9, wherein the I-OnuI HE variant has a TM₅₀ at least 15° C. higher than the TM₅₀ of the parent I-OnuI HE.

12. The I-OnuI HE variant of any one of claims 1 to 9, wherein the I-OnuI HE variant has a TM₅₀ at least 20° C. higher than the TM₅₀ of the parent I-OnuI HE.

13. The I-OnuI HE variant of any one of claims 1 to 9, wherein the I-OnuI HE variant has a TM₅₀ at least 25° C. higher than the TM₅₀ of the parent I-OnuI HE.

14. The I-OnuI HE variant of any one of claims 1 to 13, wherein the I-OnuI HE variant targets a site in a gene selected from the group consisting of: HBA, HBB, HBG1, HBG2, BCL11A, PCSK9, TCRA, TCRB, B2M, HLA-A, HLA-B, HLA-C, HLA-E, HLA-F, HLA-G, CIITA, AHR, PD-1, CTLA4, TIGIT, TGFBR2, LAG-3, TIM-3, BTLA, IL4R, IL6R, CXCR1, CXCR2, IL10R, IL13Ra2, TRAILR1, RCASIR, and FAS.

15. An I-OnuI homing endonuclease (HE) variant comprising one or more amino acid substitutions relative to a parent I-OnuI HE sequence, wherein the one or more amino acid substitutions increase the thermostability of the I-OnuI HE variant compared to the parent I-OnuI HE.

16. The I-OnuI HE variant of claim 15, wherein the parent I-OnuI HE amino acid sequence set is forth in SEQ ID NO: 1.

17. The I-OnuI HE variant of claim 15 or claim 16, wherein the one or more amino acid substitutions is at an amino acid position selected from the group consisting of: I14, A19, V116, F168, D208, N246, and L263.

18. The I-OnuI HE variant of any one of claims 15 to 17, wherein the I-OnuI HE variant comprises amino acid substitutions at the following amino acid positions: I14, A19, F168, D208, and N246.

19. The I-OnuI HE variant of claim 17 or claim 18, wherein the amino acid substituted for I14 is selected from the group consisting of: S, N, M, K, F, D, T, and V.

20. The I-OnuI HE variant of claim 17 or claim 18, wherein the amino acid substituted for I14 is selected from the group consisting of: T and V.

21. The I-OnuI HE variant of claim 17 or claim 18, wherein the amino acid substituted for A19 is selected from the group consisting of: C, D, I, L, S, T and V.

22. The I-OnuI HE variant of claim 17 or claim 18, wherein the amino acid substituted for A19 is selected from the group consisting of: T and V.

23. The I-OnuI HE variant of claim 17, wherein the amino acid substituted for V116 is selected from the group consisting of: F, D, A, L and I.

24. The I-OnuI HE variant of claim 17, wherein the amino acid substituted for V116 is selected from the group consisting of: L and I.

25. The I-OnuI HE variant of claim 17 or claim 18, wherein the amino acid substituted for F168 is selected from the group consisting of: H, Y, I, V, P, L and S.

26. The I-OnuI HE variant of claim 17 or claim 18, wherein the amino acid substituted for F168 is selected from the group consisting of: L and S.

27. The I-OnuI HE variant of claim 17 or claim 18, wherein the amino acid substituted for D208 is selected from the group consisting of: N, V, Y, and E.

28. The I-OnuI HE variant of claim 17 or claim 18, wherein the amino acid substituted for D208 is E.

29. The I-OnuI HE variant of claim 17 or claim 18, wherein the amino acid substituted for N246 is selected from the group consisting of: H, I, D, R, S, T, V, Y, and K.

30. The I-OnuI HE variant of claim 17 or claim 18, wherein the amino acid substituted for N246 is K.

31. The I-OnuI HE variant of claim 17, wherein the amino acid substituted for L263 is selected from the group consisting of: H, F, P, T, V, and R.

32. The I-OnuI HE variant of claim 17, wherein the amino acid substituted for L263 is R.

33. The I-OnuI HE variant of claim 15 or claim 16, wherein the one or more amino acid substitutions is at an amino acid position selected from the group consisting of: K108, K156, S176, E231, V261, E277, and G300.

34. The I-OnuI HE variant of claim 33, wherein the amino acid substituted for K108 is selected from the group consisting of: E, N, Q, R, T, V, and M.

35. The I-OnuI HE variant of claim 33, wherein the amino acid substituted for K108 is M

36. The I-OnuI HE variant of claim 33, wherein the amino acid substituted for K156 is selected from the group consisting of: N, Q, R, T, V, I, and E.

37. The I-OnuI HE variant of claim 33, wherein the amino acid substituted for K156 is selected from the group consisting of: I and E

38. The I-OnuI HE variant of claim 33, wherein the amino acid substituted for S176 is selected from the group consisting of: P, N and A.

39. The I-OnuI HE variant of claim 33, wherein the amino acid substituted for S176 is A.

40. The I-OnuI HE variant of claim 33, wherein the amino acid substituted for E231 is selected from the group consisting of: D, K, V, and G.

41. The I-OnuI HE variant of claim 33, wherein the amino acid substituted for E231 is selected from the group consisting of: K and G.

42. The I-OnuI HE variant of claim 33, wherein the amino acid substituted for V261 is selected from the group consisting of: D, G, I, L, S, T, and A.

43. The I-OnuI HE variant of claim 33, wherein the amino acid substituted for V261 is A.

44. The I-OnuI HE variant of claim 33, wherein the amino acid substituted for E277 is selected from the group consisting of: A, D, G, Q, V, and K.

45. The I-OnuI HE variant of claim 33, wherein the amino acid substituted for E277 is K.

46. The I-OnuI HE variant of claim 33, wherein the amino acid substituted for G300 is selected from the group consisting of: S, V, D, C, and R.

47. The I-OnuI HE variant of claim 33, wherein the amino acid substituted for G300 is R.

48. The I-OnuI HE variant of claim 15 or claim 16, wherein the one or more amino acid substitutions is at an amino acid position selected from the group consisting of: N31, N33, K52, Y97, K124, K147, I153, K209, E264, and D268.

49. The I-OnuI HE variant of claim 48, wherein the amino acid substituted for N31 is selected from the group consisting of: D, H, I, R, K, S, T and Y.

50. The I-OnuI HE variant of claim 48, wherein the amino acid substituted for N31 is K.

51. The I-OnuI HE variant of claim 48, wherein the amino acid substituted for N33 is selected from the group consisting of: D, G, H, I, K, S, T and Y.

52. The I-OnuI HE variant of claim 48, wherein the amino acid substituted for N33 is K.

53. The I-OnuI HE variant of claim 48, wherein the amino acid substituted for K52 is selected from the group consisting of: Q, R, T, Y, N, E, and M.

54. The I-OnuI HE variant of claim 48, wherein the amino acid substituted for K52 is M.

55. The I-OnuI HE variant of claim 48, wherein the amino acid substituted for Y97 is selected from the group consisting of: F, N and H.

56. The I-OnuI HE variant of claim 48, wherein the amino acid substituted for Y97 is F.

57. The I-OnuI HE variant of claim 48, wherein the amino acid substituted for K124 is selected from the group consisting of: E, N, R and T.

58. The I-OnuI HE variant of claim 48, wherein the amino acid substituted for K124 is N.

59. The I-OnuI HE variant of claim 48, wherein the amino acid substituted for K147 is selected from the group consisting of: E, I, N, R and T.

60. The I-OnuI HE variant of claim 48, wherein the amino acid substituted for K147 is I.

61. The I-OnuI HE variant of claim 48, wherein the amino acid substituted for 1153 is selected from the group consisting of: D, H, K, T, Y, S, V and N.

62. The I-OnuI HE variant of claim 48, wherein the amino acid substituted for 1153 is N.

63. The I-OnuI HE variant of claim 48, wherein the amino acid substituted for K209 is selected from the group consisting of: E, M, N, Q and R.

64. The I-OnuI HE variant of claim 48, wherein the amino acid substituted for K209 is R.

65. The I-OnuI HE variant of claim 48, wherein the amino acid substituted for E264 is selected from the group consisting of: A, D, G, K, Q, R and V.

66. The I-OnuI HE variant of claim 48, wherein the amino acid substituted for E264 is K.

67. The I-OnuI HE variant of claim 48, wherein the amino acid substituted for D268 is selected from the group consisting of: A, E, G, H, N, V and Y.

68. The I-OnuI HE variant of claim 48, wherein the amino acid substituted for D268 is N.

69. The I-OnuI HE variant of any one of claims 15 to 68, wherein the I-OnuI HE variant comprises three or more amino acid substitutions.

70. The I-OnuI HE variant of any one of claims 15 to 68, wherein the I-OnuI HE variant comprises five or more amino acid substitutions.

71. The I-OnuI HE variant of claim 15 or claim 16, wherein the I-OnuI HE variant comprises three or more amino acid substitutions is at an amino acid position selected from the group consisting of: I14, A19, K108, V116, K156, F168, S176, D208, E231, N246, V261, L263, E277, and G300.

72. The I-OnuI HE variant of claim 15 or claim 16, wherein the I-OnuI HE variant comprises three or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

73. The I-OnuI HE variant of claim 15 or claim 16, wherein the I-OnuI HE variant comprises five or more amino acid substitutions is at an amino acid position selected from the group consisting of: I14, A19, K108, V116, K156, F168, S176, D208, E231, N246, V261, L263, E277, and G300.

74. The I-OnuI HE variant of claim 15 or claim 16, wherein the I-OnuI HE variant comprises five or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

75. The I-OnuI HE variant of any one of claims 15 to 74, wherein the I-OnuI HE variant has a TM₅₀ at least 10° C. higher than the TM₅₀ of the parent I-OnuI HIE.

76. The I-OnuI HE variant of any one of claims 15 to 74, wherein the I-OnuI HE variant has a TM₅₀ at least 15° C. higher than the TM₅₀ of the parent I-OnuI HE.

77. The I-OnuI HE variant of any one of claims 15 to 74, wherein the I-OnuI HE variant has a TM₅₀ at least 20° C. higher than the TM₅₀ of the parent I-OnuI HE.

78. The I-OnuI HE variant of any one of claims 15 to 74, wherein the I-OnuI HE variant has a TM₅₀ at least 25° C. higher than the TM₅₀ of the parent I-OnuI HE.

79. The I-OnuI HE variant of any one of claims 15 to 78, wherein the I-OnuI HE variant targets a site in a gene selected from the group consisting of: HBA, HBB, HBG1, HBG2, BCL11A, PCSK9, TCRA, TCRB, B2M, HLA-A, HLA-B, HLA-C, HLA-E, HLA-F, HLA-G, CIITA, AHR, PD-1, CTLA4, TIGIT, TGFBR2, LAG-3, TIM-3, BTLA, IL4R, IL6R, CXCR1, CXCR2, IL10R, IL13Ra2, TRAILR1, RCASIR, and FAS.

80. An I-OnuI homing endonuclease (HE) variant that cleaves a target site in the human BCL11A gene, comprising one or more amino acid substitutions relative to a parent I-OnuI HE sequence, wherein the one or more amino acid substitutions increase the thermostability of the I-OnuI HE variant compared to the parent I-OnuI HE.

81. An I-OnuI homing endonuclease (HE) variant that cleaves a target site in the human PCSK9 gene, comprising one or more amino acid substitutions relative to a parent I-OnuI HE sequence, wherein the one or more amino acid substitutions increase the thermostability of the I-OnuI HE variant compared to the parent I-OnuI HE.

82. An I-OnuI homing endonuclease (HE) variant that cleaves a target site in the human PDCD-1 gene, comprising one or more amino acid substitutions relative to a parent I-OnuI HE sequence, wherein the one or more amino acid substitutions increase the thermostability of the I-OnuI HE variant compared to the parent I-OnuI HE.

83. An I-OnuI homing endonuclease (HE) variant that cleaves a target site in the human TCRα gene, comprising one or more amino acid substitutions relative to a parent I-OnuI HE sequence, wherein the one or more amino acid substitutions increase the thermostability of the I-OnuI HE variant compared to the parent I-OnuI HE.

84. An I-OnuI homing endonuclease (HE) variant that cleaves a target site in the human CBLB gene, comprising one or more amino acid substitutions relative to a parent I-OnuI HE sequence, wherein the one or more amino acid substitutions increase the thermostability of the I-OnuI HE variant compared to the parent I-OnuI HE.

85. An I-OnuI homing endonuclease (HE) variant that cleaves a target site in the human CTLA-4 gene, comprising one or more amino acid substitutions relative to a parent I-OnuI HE sequence, wherein the one or more amino acid substitutions increase the thermostability of the I-OnuI HE variant compared to the parent I-OnuI HE.

86. An I-OnuI homing endonuclease (HE) variant that cleaves a target site in the human TGFBRII gene, comprising one or more amino acid substitutions relative to a parent I-OnuI HE sequence, wherein the one or more amino acid substitutions increase the thermostability of the I-OnuI HE variant compared to the parent I-OnuI HE.

87. An I-OnuI homing endonuclease (HE) variant that cleaves a target site in the human TIM3 gene, comprising one or more amino acid substitutions relative to a parent I-OnuI HE sequence, wherein the one or more amino acid substitutions increase the thermostability of the I-OnuI HE variant compared to the parent I-OnuI HE.

88. The I-OnuI HE variant of any one of claims 80 to 87, wherein the I-OnuI HE variant comprises three or more amino acid substitutions is at an amino acid position selected from the group consisting of: I14, A19, K108, V116, K156, F168, S176, D208, E231, N246, V261, L263, E277, and G300.

89. The I-OnuI HE variant of any one of claims 80 to 87, wherein the I-OnuI HE variant comprises amino acid substitutions at the following amino acid positions: I14, A19, F168, D208, and N246.

90. The I-OnuI HE variant of any one of claims 80 to 87, wherein the I-OnuI HE variant comprises three or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

91. The I-OnuI HE variant of any one of claims 80 to 87, wherein the I-OnuI HE variant comprises five or more amino acid substitutions is at an amino acid position selected from the group consisting of: I14, A19, K108, V116, K156, F168, S176, D208, E231, N246, V261, L263, E277, and G300.

92. The I-OnuI HE variant of any one of claims 80 to 87, wherein the I-OnuI HE variant comprises five or more amino acid substitutions is at an amino acid position selected from the group consisting of: I14, A19, K108, V116, K156, F168, S176, D208, E231, N246, V261, L263, E277, and G300; and one or more amino acid substitutions is at an amino acid position selected from the group consisting of: N31, N33, K52, Y97, K124, K147, I153, K209, E264, and D268.

93. The I-OnuI HE variant of any one of claims 80 to 92, wherein the I-OnuI HE variant has a TM₅₀ at least 10° C. higher than the TM₅₀ of the parent I-OnuI HE.

94. The I-OnuI HE variant of any one of claims 80 to 92, wherein the I-OnuI HE variant has a TM₅₀ at least 15° C. higher than the TM₅₀ of the parent I-OnuI HE.

95. The I-OnuI HE variant of any one of claims 80 to 92, wherein the I-OnuI HE variant has a TM₅₀ at least 20° C. higher than the TM₅₀ of the parent I-OnuI HE.

96. The I-OnuI HE variant of any one of claims 80 to 92, wherein the I-OnuI HE variant has a TM₅₀ at least 25° C. higher than the TM₅₀ of the parent I-OnuI HE.


SEQUENCE LISTING (59 sequences)

SEQ ID NO: 1 - protein, 303 aa, Ophiostoma novo-ulmi
MAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 2 - protein, 303 aa, Ophiostoma novo-ulmi
MAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 3 - protein, 303 aa, Ophiostoma novo-ulmi
  VARIANT 1..3: note = Any amino acid or absent
XXXMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 4 - protein, 303 aa, Ophiostoma novo-ulmi
  VARIANT 1..4: note = Any amino acid or absent
  VARIANT 302..303: note = Any amino acid or absent
XXXXSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRXX

SEQ ID NO: 5 - protein, 303 aa, Ophiostoma novo-ulmi
  VARIANT 1..8: note = Any amino acid or absent
  VARIANT 302..303: note = Any amino acid or absent
XXXXXXXXSINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRXX

SEQ ID NO: 6 - protein, 301 aa, synthetic construct
  REGION 1..301: note = Made in Lab - reprogrammed I-OnuI LHE variant
  VARIANT 1..8: note = Any amino acid or absent
XXXXXXXXSINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGDGHFGVNLKKVKGTAKVYVGLRFAISQHIRDKNLMNSLITYLGCGSIREKNKSEFRWLEFEVTKFSDINDKIIPVFQENTLIGVKLEDFEDWCKVAKLIEEKKHLTESGLDEIKKIKLNMNKGR

SEQ ID NO: 7 - protein, 301 aa, synthetic construct
  REGION 1..301: note = Made in Lab - reprogrammed I-OnuI LHE variant
  VARIANT 1..8: note = Any amino acid or absent
XXXXXXXXSINPWILTGFADAEGCFRLDIRNANDLRAGYRTRLAFEIVLHNKDKSILENIQSTWKVGTIYNAGDNAVRLQVTRFEDLKVIIDHFEKYPLITQKLGDYKLFKQAFSVMENKEHLKENGIKELVRIKAKMNWGLNDELKKAFPENISKERSLINKNIPNLKWLAGFTSGDGSFVVELKKRRSPVKVGVRLRFSITQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGR

SEQ ID NO: 8 - protein, 301 aa, synthetic construct
  REGION 1..301: note = Made in Lab - reprogrammed I-OnuI LHE variant
  VARIANT 1..8: note = Any amino acid or absent
XXXXXXXXSINPWILTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGR

SEQ ID NO: 9 - protein, 301 aa, synthetic construct
  REGION 1..301: note = Made in Lab - reprogrammed I-OnuI LHE thermostable variant
  VARIANT 1..8: note = Any amino acid or absent
XXXXXXXXSINPWILTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNSKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGR

SEQ ID NO: 10 - protein, 301 aa, synthetic construct
  REGION 1..301: note = Made in Lab - reprogrammed I-OnuI LHE thermostable variant
  VARIANT 1..8: note = Any amino acid or absent
XXXXXXXXSINPWTLTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGR

SEQ ID NO: 11 - protein, 301 aa, synthetic construct
  REGION 1..301: note = Made in Lab - reprogrammed I-OnuI LHE thermostable variant
  VARIANT 1..8: note = Any amino acid or absent
XXXXXXXXSINPWILTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDIIDKIIPVFQENTLIGVKLEDFEDWCKVAKLIEEKKHLTESGLDEIKKIKLNMNKGR

SEQ ID NO: 12 - protein, 301 aa, synthetic construct
  REGION 1..301: note = Made in Lab - reprogrammed I-OnuI LHE thermostable variant
  VARIANT 1..8: note = Any amino acid or absent
XXXXXXXXSINPWILTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGAKLEDFEDWCKVAKLIEEKKHLTESGLDEIKKIKLNMNKGR

SEQ ID NO: 13 - protein, 301 aa, synthetic construct
  REGION 1..301: note = Made in Lab - reprogrammed I-OnuI LHE thermostable variant
  VARIANT 1..8: note = Any amino acid or absent
XXXXXXXXSINPWILTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVRLVFEISQHVRDKNLMNSLITYLGCGHIYEGNKSERSWLQFRVEKFSDIIDKIIPVFQENTLIGAKHEDFEDWCKVAKLIEEKKHLTESGLDEIKKIKLNMNKGR

SEQ ID NO: 14 - protein, 301 aa, synthetic construct
  REGION 1..301: note = Made in Lab - reprogrammed I-OnuI LHE thermostable variant
  VARIANT 1..8: note = Any amino acid or absent
XXXXXXXXSINPWTLTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPEVISRERPLINKNIPNGKWLAGFTSGDGSFFVRLRKSNVNARVRVQLVFEISQHIRDKNLMNSLITYLGCGHIYEGNKSERSWLQFRVEKFSDINDKIIPVFQENTLIGMKLEDFEDWCKVAKLIEEKKHLTESGLDEIKKIKLNMNKRR

SEQ ID NO: 15 - protein, 301 aa, synthetic construct
  REGION 1..301: note = Made in Lab - reprogrammed I-OnuI LHE variant
  VARIANT 1..8: note = Any amino acid or absent
XXXXXXXXSINPWILTGFADAEGSFGLSILNRNRGTARYHTRLSFTIMLHNKDKSILENIQSTWKVGSILNNGDHYVSLVVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGR

SEQ ID NO: 16 - protein, 301 aa, synthetic construct
  REGION 1..301: note = Made in Lab - reprogrammed I-OnuI LHE thermostable variant
  VARIANT 1..8: note = Any amino acid or absent
XXXXSRRESINPWTLTGFADAEGSFGLSILNRNRGTARYHTRLSFTIMLHNKDKSILENIQSTWKVGSILNNGDHYVSLVVYAFEDLKVIIDHFEKYPLITQKLGDYKLFKQAFSVMENKEHLKENGIKELVRIKAKMNWGLNDELKKAFPEVISRERPLINKNIPNGKWLAGFTSGDGSFFVRLRKSNVNARVRVQLVFEISQHIRDKNLMNSLITYLGCGHIYEGNKSERSWLQFRVEKFSDINDKIIPVFQENTLIGMKLEDFEDWCKVAKLIEEKKHLTESGLDEIKKIKLNMNKRR

SEQ ID NO: 17 - protein, 301 aa, synthetic construct
  REGION 1..301: note = Made in Lab - reprogrammed I-OnuI LHE thermostable variant
  VARIANT 1..8: note = Any amino acid or absent
XXXXXXXXSINPWTLTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPEVISRERPLINKNIPNGKWLAGFTAGDGHFGVNLKKVKGTAKVYVGLRFAISQHIRDKNLMNSLITYLGCGSIREKNKSEFRWLEFEVTKFSDINDKIIPVFQENTLIGMKLEDFEDWCKVAKLIEEKKHLTESGLDEIKKIKLNMNKRR

SEQ ID NO: 18 - protein, 301 aa, synthetic construct
  REGION 1..301: note = Made in Lab - reprogrammed I-OnuI LHE thermostable variant
  VARIANT 1..8: note = Any amino acid or absent
XXXXXXXXSINPWTLTGFADAEGCFRLDIRNANDLRAGYRTRLAFEIVLHNKDKSILENIQSTWKVGTIYNAGDNAVRLQVTRFEDLKVIIDHFEKYPLITQKLGDYKLFKQAFSVMENKEHLKENGIKELVRIKAKMNWGLNDELKKAFPEVISRERSLINKNIPNGKWLAGFTSGDGSFVVELKKRRSPVKVGVRLRFSITQHIRDKNLMNSLITYLGCGRIVENNKSEHSWLEFIVTKFSDINDKIIPVFQENTLIGMKLEDFEDWCKVAKLIEEKKHLTESGLDEIKKIKLNMNKRR

SEQ ID NO: 19 - RNA, 1743 nt, synthetic construct
  misc_feature 1..1743: note = Made in Lab - BCL11A I-OnuI HE variant codon optimized mRNA
  source 1..1743: mol_type = other RNA
atgggatcaccgcctaagaagaagagaaaagtgtcgagaagggaatcaattaacccttggatcctgaccggcttcgccgacgccgaggggtcattcctcctccgcattcgcaataataacaagagtagcgtaggctactctactgagctgggcttccagattaccctccacaacaaggacaagagtatcctggaaaacatccagtcgacttggaaggtgggcgtaatagctaacagtggagacaacgctgtgtcactcaaagtcaccaggttcgaggacctgaaggtgatcattgaccacttcgagaagtacccactgatcactcagaagctgggtgactacaagctgttcaagcaagcattttcggtgatggagaacaaagagcacctgaaggaaaatggaattaaggaacttgtgcgcatcaaggccaagttgaactggggacttacggatgagctcaagaaagcgtttcctgaaaacatctcgaaagagaggagtctgattaacaagaatatccctaacttcaagtggctggccgggttcacgtccggggaaggatgctttttcgtcaacctcataaaaagcaagtctaaacttggagtgcaagtgcagctggtgttttcaatcactcaacacatcaaagacaagaacctcatgaactcgctcatcacctacctgggttgcggatatatcaaagagaaaaacaagtcagaattttcctggctggatttcgtggtgacaaagttcagcgacatcaacgacaagatcatcccggtgttccaggagaacaccctcatcggcgtgaagctggaggacttcgaagattggtgcaaggtggcgaagcttatcgaagaaaagaagcacctgaccgagtccggactggacgagatcaagaagatcaaactgaacatgaacaagggccggagatatccatacgatgtcccagattatgcgctcgagagtggggaaggtcgcggctccttgctcacctgtggggatgtcgaagaaaacccaggtcccgctagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 20 - RNA, 1743 nt, synthetic construct
  misc_feature 1..1743: note = Made in Lab - BCL11A I-OnuI HE variant mRNA
  source 1..1743: mol_type = other RNA
atgggatcaccgcctaagaagaagagaaaagtgtcgagaagggaatccatcaacccatggattctgactggtttcgctgatgccgaaggaagcttcgtgctaagtatccaaaacagaaacgattatgctactggttacagaattcacctgacattccaaatcaccctgcacaacaaggacaaatcgattctggagaatatccagtcgacttggaaggtcggcaaaatcaataacacgggcgacaacctcgtccaactgagagtctaccgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacatctggtgatggctccttcttcgtgcgcctaagaaagtctaatgttaatgctagagtacgtgtgcaactggtattcgagatctcacagcacatcagagacaagaacctgatgaattcattgataacatacctaggctgtggtcacatctacgagggaaacaaatctgagcgcagttggctccaattcagagtagaaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtagatatccatacgatgtcccagattatgcgctcgagagtggggaaggtcgcggctccttgctcacctgtggggatgtcgaagaaaacccaggtcccgctagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 21 - RNA, 1743 nt, synthetic construct
  misc_feature 1..1743: note = Made in Lab - BCL11A I-OnuI HE A5 variant mRNA
  source 1..1743: mol_type = other RNA
atgggatcaccgcctaagaagaagagaaaagtgtcgagaagggaatccatcaacccatggactctgactggtttcgctgatgccgaaggaagcttcgtgctaagtatccaaaacagaaacgattatgctactggttacagaattcacctgacattccaaatcaccctgcacaacaaggacaaatcgattctggagaatatccagtcgacttggaaggtcggcaaaatcaataacacgggcgacaacctcgtccaactgagagtctaccgtttcgaagatttgaaagtgattatcgaccacttcgagaaatatccgctgataacacagaaattgggcgattacaaattgtttaaacaggcattcagcgtcatggagaacaaagaacatcttaaggagaatgggattaaggagctcgtacgaatcaaagctaagatgaattggggtctcaatgacgaattgaaaaaagcatttccagaggtgattagcagggagcgcccccttatcaataagaacattccgaatgggaaatggctggctggattcacatctggtgatggctccttcttcgtgcgcctaagaaagtctaatgttaatgctagagtacgtgtgcaactggtattcgagatctcacagcacatcagagataagaacctgatgaattcattgataacatacctaggctgtggtcacatctacgagggaaacaaatctgagcgctcttggctccaattcagagtagaaaaattcagcgatatcaatgacaagatcattccggtattccaggaaaatactctgattggcatgaaactcgaggactttgaagattggtgcaaggttgccaaattgatcgaagagaagaaacacctgaccgaatccggtttggatgagattaagaaaatcaagctgaacatgaacaaaagacgtagatatccatacgatgtcccagattatgcgctcgagagtggggaaggtcgcggctccttgctcacctgtggggatgtcgaagaaaacccaggtcccgctagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 22 - protein, 944 aa, synthetic construct
  REGION 1..944: note = Made in Lab - engineered PDCD-1 megaTAL
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAISRVGGSSRRESINPWILTGFADAEGSFGLSILNRNRGTARYHTRLSFTIMLHNKDKSILENIQSTWKVGSILNNGDHYVSLVVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGR

SEQ ID NO: 23 - protein, 944 aa, synthetic construct
  REGION 1..944: note = Made in Lab - engineered PDCD-1 megaTAL
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAISRVGGSSRRESINPWTLTGFADAEGSFGLSILNRNRGTARYHTRLSFTIMLHNKDKSILENIQSTWKVGSILNNGDHYVSLVVYAFEDLKVIIDHFEKYPLITQKLGDYKLFKQAFSVMENKEHLKENGIKELVRIKAKMNWGLNDELKKAFPEVISRERPLINKNIPNGKWLAGFTSGDGSFFVRLRKSNVNARVRVQLVFEISQHIRDKNLMNSLITYLGCGHIYEGNKSERSWLQFRVEKFSDINDKIIPVFQENTLIGMKLEDFEDWCKVAKLIEEKKHLTESGLDEIKKIKLNMNKRR

SEQ ID NO: 24 - skipped

SEQ ID NO: 25 - protein, 5 aa, synthetic construct
  REGION 1..5: note = Exemplary linker sequence
DGGGS

SEQ ID NO: 26 - protein, 5 aa, synthetic construct
  REGION 1..5: note = Exemplary linker sequence
TGEKP

SEQ ID NO: 27 - protein, 4 aa, synthetic construct
  REGION 1..4: note = Exemplary linker sequence
GGRR

SEQ ID NO: 28 - protein, 5 aa, synthetic construct
  REGION 1..5: note = Exemplary linker sequence
GGGGS

SEQ ID NO: 29 - protein, 14 aa, synthetic construct
  REGION 1..14: note = Exemplary linker sequence
EGKSSGSGSESKVD

SEQ ID NO: 30 - protein, 18 aa, synthetic construct
  REGION 1..18: note = Exemplary linker sequence
KESGSVSSEQLAQFRSLD

SEQ ID NO: 31 - protein, 8 aa, synthetic construct
  REGION 1..8: note = Exemplary linker sequence
GGRRGGGS

SEQ ID NO: 32 - protein, 9 aa, synthetic construct
  REGION 1..9: note = Exemplary linker sequence
LRQRDGERP

SEQ ID NO: 33 - protein, 12 aa, synthetic construct
  REGION 1..12: note = Exemplary linker sequence
LRQKDGGGSERP

SEQ ID NO: 34 - protein, 16 aa, synthetic construct
  REGION 1..16: note = Exemplary linker sequence
LRQKDGGGSGGGSERP

SEQ ID NO: 35 - skipped

SEQ ID NO: 36 - protein, 7 aa, synthetic construct
  REGION 1..7: note = Cleavage sequence by TEV protease
ENLYFQG

SEQ ID NO: 37 - protein, 7 aa, synthetic construct
  REGION 1..7: note = Cleavage sequence by TEV protease
ENLYFQS

SEQ ID NO: 38 - protein, 22 aa, synthetic construct
  REGION 1..22: note = Self-cleaving polypeptide comprising 2A site
GSGATNFSLLKQAGDVEENPGP

SEQ ID NO: 39 - protein, 19 aa, synthetic construct
  REGION 1..19: note = Self-cleaving polypeptide comprising 2A site
ATNFSLLKQAGDVEENPGP

SEQ ID NO: 40 - protein, 14 aa, synthetic construct
  REGION 1..14: note = Self-cleaving polypeptide comprising 2A site
LLKQAGDVEENPGP

SEQ ID NO: 41 - protein, 21 aa, synthetic construct
  REGION 1..21: note = Self-cleaving polypeptide comprising 2A site
GSGEGRGSLLTCGDVEENPGP

SEQ ID NO: 42 - protein, 18 aa, synthetic construct
  REGION 1..18: note = Self-cleaving polypeptide comprising 2A site
EGRGSLLTCGDVEENPGP

SEQ ID NO: 43 - protein, 13 aa, synthetic construct
  REGION 1..13: note = Self-cleaving polypeptide comprising 2A site
LLTCGDVEENPGP

SEQ ID NO: 44 - protein, 23 aa, synthetic construct
  REGION 1..23: note = Self-cleaving polypeptide comprising 2A site
GSGQCTNYALLKLAGDVESNPGP

SEQ ID NO: 45 - protein, 20 aa, synthetic construct
  REGION 1..20: note = Self-cleaving polypeptide comprising 2A site
QCTNYALLKLAGDVESNPGP

SEQ ID NO: 46 - protein, 14 aa, synthetic construct
  REGION 1..14: note = Self-cleaving polypeptide comprising 2A site
LLKLAGDVESNPGP

SEQ ID NO: 47 - protein, 25 aa, synthetic construct
  REGION 1..25: note = Self-cleaving polypeptide comprising 2A site
GSGVKQTLNFDLLKLAGDVESNPGP

SEQ ID NO: 48 - protein, 22 aa, synthetic construct
  REGION 1..22: note = Self-cleaving polypeptide comprising 2A site
VKQTLNFDLLKLAGDVESNPGP

SEQ ID NO: 49 - protein, 14 aa, synthetic construct
  REGION 1..14: note = Self-cleaving polypeptide comprising 2A site
LLKLAGDVESNPGP

SEQ ID NO: 50 - protein, 19 aa, synthetic construct
  REGION 1..19: note = Self-cleaving polypeptide comprising 2A site
LLNFDLLKLAGDVESNPGP

SEQ ID NO: 51 - protein, 19 aa, synthetic construct
  REGION 1..19: note = Self-cleaving polypeptide comprising 2A site
TLNFDLLKLAGDVESNPGP

SEQ ID NO: 52 - protein, 14 aa, synthetic construct
  REGION 1..14: note = Self-cleaving polypeptide comprising 2A site
LLKLAGDVESNPGP

SEQ ID NO: 53 - protein, 17 aa, synthetic construct
  REGION 1..17: note = Self-cleaving polypeptide comprising 2A site
NFDLLKLAGDVESNPGP

SEQ ID NO: 54 - protein, 20 aa, synthetic construct
  REGION 1..20: note = Self-cleaving polypeptide comprising 2A site
QLLNFDLLKLAGDVESNPGP

SEQ ID NO: 55 - protein, 24 aa, synthetic construct
  REGION 1..24: note = Self-cleaving polypeptide comprising 2A site
APVKQTLNFDLLKLAGDVESNPGP

SEQ ID NO: 56 - protein, 40 aa, synthetic construct
  REGION 1..40: note = Self-cleaving polypeptide comprising 2A site
VTELLYRMKRAETYCPRPLLAIHPTEARHKQKIVAPVKQT

SEQ ID NO: 57 - protein, 18 aa, synthetic construct
  REGION 1..18: note = Self-cleaving polypeptide comprising 2A site
LNFDLLKLAGDVESNPGP

SEQ ID NO: 58 - protein, 40 aa, synthetic construct
  REGION 1..40: note = Self-cleaving polypeptide comprising 2A site
LLAIHPTEARHKQKIVAPVKQTLNFDLLKLAGDVESNPGP

SEQ ID NO: 59 - protein, 33 aa, synthetic construct
  REGION 1..33: note = Self-cleaving polypeptide comprising 2A site
EARHKQKIVAPVKQTLNFDLLKLAGDVESNPGP